(19) United States
(12) Patent Application Publication
(10) Pub. No.: US 2024/0005815 A1
(43) Pub. Date: Jan. 4, 2024

(54) Nutrition Management System and Method

(71) Applicant: EVOLT IP PTY LTD, Burleigh Heads, Queensland (AU)

(72) Inventors: Edward Jason Zouroudis, Mermaid Waters, Queensland (AU); Kylie Marie Zimmerle, Tamborine, Queensland (AU)

(21) Appl. No.: 18/035,457

(22) PCT Filed: Nov. 5, 2021
(86) PCT No.: PCT/AU2021/051312

§ 371 Date: May 4, 2023

(30) Foreign Application Priority Data
Nov. 6, 2020 (AU) 2020904054

(51) Int. Cl. G09B 19/00; G16H 20/60; A61B 5/00; A61B 5/0537

(52) U.S. Cl. CPC G09B 19/0092; G16H 20/60; A61B 5/4866; A61B 5/0537

(57) ABSTRACT

A method for providing a personalised caloric and/or macronutrient intake profile for a user is disclosed. In an embodiment, the method comprises obtaining a set of values of biological characteristics for the user, wherein the set includes at least one value derived from sensed information, wherein the at least one value includes a value of an anthropometric measurement and/or a value of a bio-impedance measurement. A basal metabolic rate (BMR) estimation for the user is then determined based on the set of values of biological characteristic and an energy requirement estimation is determined based on at least the basal metabolic rate (BMR) estimation; and determining the personalised caloric and/or macronutrient intake profile for the user based on at least the energy requirement estimation.

### TECHNICAL FIELD

The present disclosure relates to a system and method for providing nutrition and diet information based on a user's anticipated caloric needs and/or lifestyle.

### BACKGROUND

In today's society, there is increasing interest in being able to match one's diet with their lifestyle requirements so as to assist with, for example, managing weight or ensuring that their energy intake meets the energy demands of their lifestyle. Often this involves determining one's caloric requirements and then managing caloric intake accordingly by dieting.

Platforms exist which allow a user to determine their basic caloric needs based on, for example, their height, age, gender and activity level. For example, a user may use an online platform to determine a basic caloric need and then use another platform to determine macronutrient values. Using such an approach, having determined a basic caloric need, a user could then access another platform to create a dietary prescription based on the determined caloric (and potentially macronutrient) values as well as potentially find a suitable supplementation protocol.

In another approach, a user may obtain body composition information in a clinical or commercial setting by performing a scan, such as a “3D body scan”, which estimates the user's percentage body fat. As a part of such an approach, the user may be also required to provide their age, gender, height and weight as input into an algorithm and provide external measurements and estimates of, for example, body composition volume based on normative values. In a traditional 3D body scan setting, the user experience ends with the metrics it provides. However, there is no additional calculation conducted to determine, validate or quantify the overall health status of the user, let alone determine a dietary prescription.

One attempt to provide a diet prescription to users based on personalised health characteristics is disclosed in U.S. Pat. No. 5,542,420. This patent describes a system for prescribing a suitable diet to users based on their personalised health characteristics. Information about food consumed and medical data is entered at terminals and communicated to a ‘health computer’ which then determines the dietary prescription. However, the health characteristics used to prescribe the diet to a user are limited.

It would be desirable to provide an improved method and system for providing a personalised caloric and/or macronutrient intake profile for a user.

### SUMMARY

One or more embodiments of the present disclosure provide a system and/or method for providing a personalised caloric and/or macronutrient intake profile for a user which depends on an estimated energy requirement for the user.

According to a first aspect of the present disclosure there is provided a method of providing a personalised caloric and/or macronutrient intake profile for a user, comprising:
obtaining an energy requirement estimation based on a basal metabolic rate (BMR) estimation for the user; and
determining the personalised caloric and/or macronutrient intake profile for the user based on the energy requirement estimation.

According to a second aspect of the present disclosure, there is provided a method of providing a personalised caloric and/or macronutrient intake profile for a user, comprising:
obtaining a value of one or more biological characteristics for the user;
obtaining an energy requirement estimation based on a determined basal metabolic rate (BMR) estimation, said determined basal metabolic rate estimation depending on the one or more values of biological characteristics; and
processing the estimated energy requirement and one or more biological characteristics associated with the user to select the personalised caloric and/or nutrient intake profile for the user.

According to a third aspect of the present disclosure there is provided a method of providing a personalised caloric and/or macronutrient intake profile for a user, comprising:
obtaining a set of values of biological characteristics for the user, wherein the set includes at least one value derived from sensed information, wherein the at least one value includes a value of an anthropometric measurement and/or a value of a bio-impedance measurement;
determining a basal metabolic rate (BMR) estimation for the user based on the set of values of biological characteristics;
determining an energy requirement estimation based on at least the basal metabolic rate (BMR) estimation; and
determining the personalised caloric and/or macronutrient intake profile for the user based on at least the energy requirement estimation.

According to yet another aspect of an embodiment of the disclosure there is provided a method for providing a personalised caloric and/or macronutrient intake profile for a user, comprising:
obtaining a set of values of biological characteristics for the user, wherein the set includes at least one value derived from sensed information, wherein the at least one value includes a value of an anthropometric measurement and/or a value of a bio-impedance measurement;
assigning a body shape classification attribute to the user based on one or more of the values of biological characteristics of the set;
retrieving a template including plural fields for caloric parameters and/or macronutrient parameters and/or expressions, said retrieved template depending on the body shape classification attribute;
determining a basal metabolic rate (BMR) estimation for the user based on the set of values of biological characteristics;
determining an energy requirement estimation based on at least the basal metabolic rate (BMR) estimation; and
determining the personalised caloric and/or macronutrient intake profile for the user based on at least the energy requirement estimation,
wherein determining the personalised caloric and/or macronutrient intake profile for the user based on at least the energy requirement estimation comprises populating the retrieved template with caloric and/or macronutrient values, or ranges of such values, depending on the estimated energy requirement.

In an embodiment, obtaining a value of one or more biological characteristics for the user includes:
obtaining one or more images of the user's body;
processing the one or more images to derive the one or more values of biological as one or more of the user's height, weight, age and/or gender.

According to another aspect of an embodiment of the present disclosure there is provided a system for providing a personalised caloric and/or macronutrient intake profile for a user, comprising:
input means for obtaining a set of values of biological characteristics for the user, wherein the set includes at least one value derived from sensed information, wherein the at least one value includes a value of an anthropometric measurement and/or a value of a bio-impedance measurement; and
processing means for:
   determining a basal metabolic rate (BMR) estimation for the user based on the set of values of biological characteristics;
   processing means for determining an energy requirement estimation based on at least the basal metabolic rate (BMR) estimation; and
   processing the estimated energy requirement to select the personalised caloric and/or macronutrient intake profile for the user.

According to yet another aspect of an embodiment of the present disclosure there is provided a system for generating a personalised caloric and/or macronutrient intake profile for a user, comprising:
a user device for receiving input information including:
   information from which the user's body shape, height, weight, age, and gender of may be obtained or derived; and
   one or more activity attributes classifying a dominant physical activity level and/or activity type for the user;
a host server in data communication with the user device to:
   receive the input information;
   process the input information to:
      assign a body shape classification attribute to the user;
      retrieve a template including plural fields for caloric parameters and/or macronutrient parameters, said retrieved template depending on the body shape classification attribute and the one or more activity attributes;
      determine a base basal metabolic rate (BMR) estimation for the user based on the derived or obtained height, weight, age, and gender information;
      determine an energy requirement estimation based on at least the determined base basal metabolic rate (BMR) and the one or more activity attributes;
      generate the personalised caloric and/or macronutrient intake profile for the user based on at least the energy requirement estimation, wherein generating the personalised caloric and/or macronutrient intake profile for the user based on at least the energy requirement estimation comprises populating the retrieved template with caloric and/or macronutrient values, or ranges of such values, depending on the estimated energy requirement; and
      communicate the determined personalised caloric and/or macronutrient intake profile to the user device.

According to another aspect of an embodiment of the present disclosure there is provided a computer readable media including a set of program instructions which are executable by one or more processors to:
obtaining a value of one or more biological characteristics for the user
obtaining an energy requirement estimation based on a determined basal metabolic rate (BMR) estimation, said determined basal metabolic rate estimation depending on the one or more values of biological characteristics; and
processing the estimated energy requirement and one or more biological characteristics associated with the user to select the personalised caloric and/or nutrient intake profile for the user.

Embodiments of the present disclosure may involve a user operating a user device hosting a user interface, as an when required, to generate as an output a personalised caloric and/or nutrient intake profile.

Furthermore, embodiments which obtain or derive a user's biological and/or anthropometric information via the use of an image capturing device, and which then use that information to generate, as an output, the personalised caloric and/or nutrient intake profile are expected to allow for simplified generation of the personalised caloric and/or nutrient intake profile with a reduced reliance on obtaining physical measurements, thus also reducing a reliance on access to and availability of instruments and devices required to obtain those measurements.

### BRIEF DESCRIPTION OF DRAWINGS

Embodiments of the present disclosure will be discussed with reference to the accompanying drawings wherein:

FIG. 1A is a block diagram of a system according to an embodiment of the present disclosure;

FIG. 1B is a block diagram of a system according to another embodiment of the present disclosure;

FIG. 2 is a flow diagram of a method according to an embodiment of the present disclosure;

FIG. 3 is a flow diagram of an approach for processing an energy requirement estimation and one or more classification attributes suitable for incorporating in the method of FIG. 2;

FIG. 4 is a flow diagram of an example approach for obtaining a body shape classification attribute suitable for incorporating in the approach of FIG. 3;

FIG. 5 is a flow diagram of another example approach for obtaining a body shape classification attribute suitable for incorporating in the approach of FIG. 3;

FIG. 6 is a flow diagram of another example approach for obtaining a body shape classification attribute suitable for incorporating in the approach of FIG. 3;

FIG. 7 is a flow diagram of an example approach for determining a caloric and/or macronutrient profile suitable for incorporating in the method of FIG. 2;

FIG. 8A to FIG. 8C are functional block diagrams of a system for generating a caloric and/or macronutrient profile for a user according to an embodiment;

FIG. 9 is a functional block diagram of another system for generating a caloric and/or macronutrient profile for a user according to an embodiment;

FIG. 10 is a series of user interfaces suitable for use with a system embodiment for generating a caloric and/or macronutrient profile for a user according to an embodiment;

FIG. 11 is a series of user interfaces suitable for use with a system embodiment for generating a caloric and/or macronutrient profile for a user according to an embodiment;

FIG. 12 is a series of user interfaces suitable for use with a system embodiment for generating a caloric and/or macronutrient profile for a user according to an embodiment;

FIG. 13 is a sequence of user interfaces suitable for use with a system embodiment for generating a caloric and/or macronutrient profile for a user according to an embodiment;

FIG. 14A to FIG. 14C are example caloric and/or macronutrient profiles generated by an embodiment of the present disclosure;

FIG. 15A to FIG. 15F are example caloric and/or macronutrient profiles generated by an embodiment of the present disclosure;

FIG. 16A to FIG. 16R are example caloric and/or macronutrient profiles generated by an embodiment of the present disclosure;

FIGS. 17 and 18 are flow diagrams of example processes in accordance with some embodiments; and

FIG. 19 is a block diagram of a computing system suitable for use with an embodiment of the present disclosure.

In the following description, like reference characters designate like or corresponding parts throughout the figures.

### DESCRIPTION OF EMBODIMENTS

Preferred features of the present invention will now be described with particular reference to the accompanying drawings. However, it is to be understood that the features illustrated in and described with reference to the drawings are not to be construed as limiting on the scope of the invention.

A system and method according to embodiments of the present disclosure will be described below in relation to an application for providing, for a user, a personalised caloric and/or macronutrient profile which involves obtaining a set of one or more values of biological characteristics for the user and determining a basal metabolic rate (BMR) estimation for the user based on the set of one or more obtained values. An estimated energy requirement for the user is then determined using at least the basal metabolic rate (BMR) estimation. The estimated energy is then used to select the personalised caloric and/or macronutrient intake profile for the user.

In embodiments, at least one value of the set of values includes an anthropometric measurement or a value of a bio-impedance measurement derived from a sensed information obtained from a suitable sensor or device.

One example, of a suitable device is the “Evolt 360 Bioelectrical Impedance Analysis (BIA) machine” which provides an 8-point, multi-frequency, segmental body composition analysis. The device uses impedance and reactance to differentiate lean body mass tissue (hydrous) from fat tissue (anhydrous). Establishing the components of lean body tissue, such as skeletal body mass, allows for the determination of the user's basal metabolic rate (BMR).

In other examples, a suitable sensor or device may include a weight sensor for obtaining a value of a weight biological characteristic for the user, or an image capturing device for obtaining a value of an anthropometric measurement, such as height or waist measurement, or indeed the user's age and/or gender.

In other words, in certain embodiments of the disclosure, a set of values of biological characteristics is obtained for the user, such that at least one of the values of the set includes a value derived from sensed information.

In embodiments, determining the basal metabolic rate estimation may involve processing the obtained set of values of biological characteristics, which may include user entered and/or sensed biological characteristics. The energy requirement estimation based on at least the basal metabolic rate estimation may be determined and processed, potentially using one or more additional user attributes and/or requirements, to obtain the personalised caloric and/or macronutrient intake profile for the user. The one or more additional user attributes and/or requirements may be entered into a device by the user or they may be derived by processing one or more of the values of set of values of biological characteristics or other sensed or entered information. Examples of additional user attributes will be described later.

In certain embodiments, providing the personalised caloric and/or macronutrient intake profile for the user involves indexing user attributes and/or requirements into a database to select a personalised caloric and/or macronutrient intake profile including a set of caloric and/or macronutrient values and/or ranges which depend on the user attributes and/or requirements and the user's BMR. In other embodiments, the personalised caloric and/or macronutrient intake profile may be constructed using the energy requirement estimation, and potentially the additional user attributes and/or requirements, as input(s) to an information processing system, such as an artificial neural network based information processing system programmed with suitable training data.

As will be described in more detail below, in certain embodiments one or more values of the set of values of biological characteristics for the user may be entered into a user device as, for example, body height and waist measurements for the user, for processing to determine the basal metabolic rate estimation for the user and potentially additional user attributes. Age and gender information may also be entered or otherwise obtained.

As described above, one or more values of biological characteristics for the user may be derived from sensed information obtained, for example, from a suitable sensor. For example, in some embodiments, one or more values of biological characteristics for the user may be derived from sensed image information obtained from an image of the user's body which has been captured by an image capturing device. Examples of values of biological characteristics which may be derived from an image of the user's body, using suitable processing and image recognition techniques, include measures of height, weight, age and gender. Techniques for deriving one or more values of biological characteristics for the user may be derived from sensed image information obtained from an image would be well understood to a person skilled in the art.

In other embodiments, one or more values of biological characteristics for the user may be derived from one or more electrical sensors in contact with the user, such as a bio-impedance sensor.

In view of the above, it will thus be appreciated that embodiments of the present disclosure contemplate various techniques for obtaining a set of values of biological characteristics for the user.

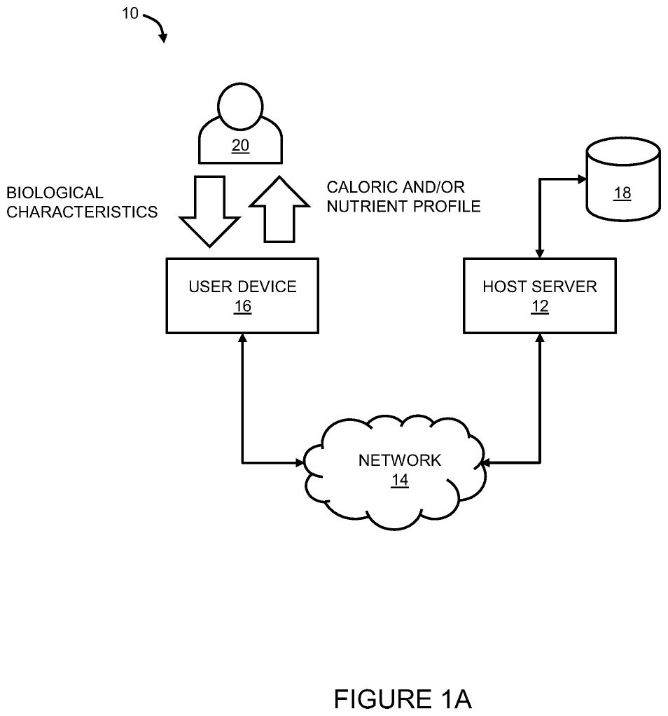

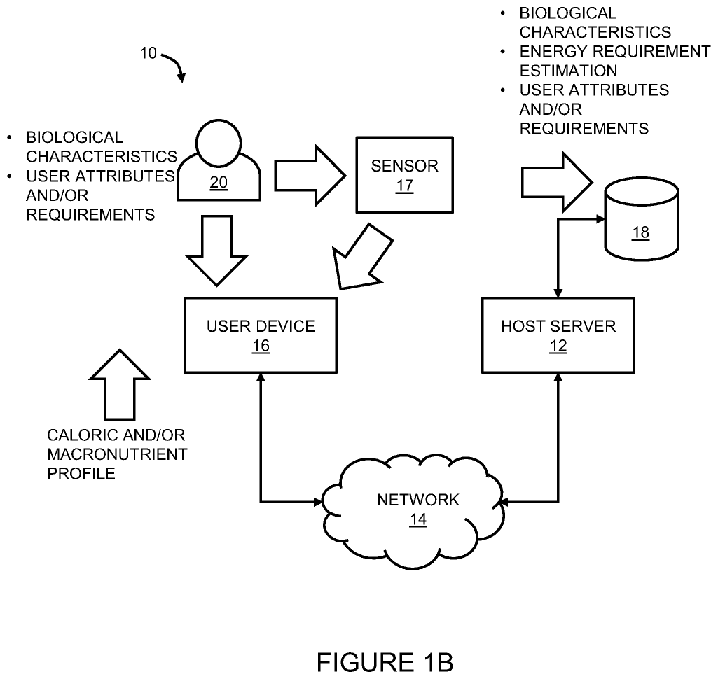

Referring now to FIG. 1, there is shown an example of a system 10 in accordance with the present disclosure. The depicted system 10 includes a network 14 capable of supporting data communication between one or more host servers 12 and one or more user devices 16 associated with a user 20. The host server 12 and user devices 16 shown here communicate with the network 14 via a suitable wired or wireless communication interface. Suitable communication interfaces will be well understood by a person skilled in the art.

In the present case, the one or more host servers 12 store and process information communicated by user devices 16, such as one or more values of biological characteristics for the user. The host servers 12 are also able to access one or more databases 18 storing data necessary for the operation of the methods and systems of the present disclosure, potentially including user attributes and/or requirements entered into and received from user devices 16 or derived from processing one or more values of biological characteristics for the user. The host servers 12 may comprise any of a number of servers known to those skilled in the art.

Each host server 12 may include a central processing unit or CPU that includes one or more microprocessors and memory operably connected to the CPU. The CPU may comprise a parallel processor, a vector processor, or a distributed computing device. The memory may include any combination of random access memory (RAM), a storage medium such as a magnetic hard disk drive(s) and the like. The memory is operatively coupled to the CPU and may comprise RAM and ROM components, and may be provided within or external to the host servers 12. The memory may be used to store the operating system and additional software modules or instructions, databases and the like. The host servers 12 may be configured to load and executed the software modules or instructions stored in the memory.

As will be discussed in more detail below, in a preferred embodiment the or each database 18 stores data relating to each user 20 of the system 10, such as one or more values of biological characteristics and potentially one or more user attributes and/or requirements. Other information may also be stored in the database(s) 18, such as relationships between the one or more values of biological characteristics for the user, estimated energy requirements, one or more of the user attributes and/or requirements, and one or more associated caloric and/or macronutrient profiles.

In a preferred embodiment, network 14 is a distributed computing network such as the internet or a dedicated mobile or cellular network in combination with the internet, such as a GSM, 3G, 4G, 5G, CDMA or WCDMA network. Other types of networks such as an intranet, an extranet, a virtual private network (VPN) and non-TCP/IP based networks are also contemplated.

The user devices 16 are typically in the form of an electronic computing device such as a desktop computer, laptop computer, tablet, smart watch, smart phone, Personal Digital Assistant (PDA) or the like, configured with a dedicated software application to assist the user 20 in entering information for use by the system and method of the present invention provide a personalised caloric and/or macronutrient profile for a user 20 based on an estimated energy requirement, such as a basal metabolic rate (BMR) estimation, and user attributes and/or requirements. Examples of suitable user attributes and/or requirements will be described below.

In embodiments, each user devices 16 stores one or more software programs including executable code to facilitate operation of a software application or “app” configured to provide an interface between the user devices 16 and the host server 12 to enable communication therebetween. The functionality of the user devices 16 is provided by the software application installed in local non-volatile storage of the user device 16 and which is executed by an internal processor of the user device 16.

As shown in FIG. 1B, each user device 16 may include or be in communication with a sensor 17, such as an imaging device (such as a camera), for obtaining an image of the body of the user 20 for processing to derive a user attribute, and/or a bio-impedance sensor to provide a value(s) of biological characteristics for the user in the form of a value of bio-impedance or a body composition report produced from such values.

The software application may be downloaded to, or installed on, the user device 16 via the network 14 from a software application store, such as iTunes® or Google Play, or indeed directly from the host sever 12. In this respect, the or each user device 16 may be configured to collect and transfer information to the host servers 12 via the network 14 automatically, or in response to a user command, by transmitting data collected by the user device 16 in a form suitable for communication between the user device 16 and the host server 12.

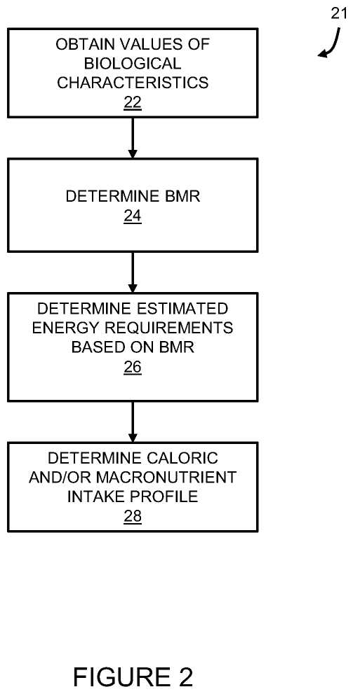

In one embodiment, in order for a user 20 to use the system 10 of the present disclosure, the user 20 may follow the method 21 as set out in FIG. 2.

In the present case, at step 22, a user, via their user device 16 operating a software application, inputs one or more values of biological characteristics which, in the present example, comprise the user's age, weight, height and gender.

Before continuing further, although in the present case one or more values of biological characteristics are input into the user device 16 at step 22, it is not essential that this be the case.

For example, in some embodiments it is possible that one or more values of biological characteristics may be obtained or determined from one or more data sources (such as from an account that the user has created or subscribed to with an external software platform or application).

In other embodiments, it is possible that one or more values of biological characteristics are obtained or determined from one or more sensors or devices capable of sensing or otherwise providing values of biological characteristics. For example, in one embodiment, a body weight biological characteristic of the user may be obtained from a weight sensor (such as a weight scale) in communication with the user device 16.

In another embodiment, values of one or more biological characteristics, such as age and gender, may be determined or estimated from image analysis of an image of the user 20. Suitable techniques for determining or estimating age and gender using image analysis of an image of the user 20 would be well understood by a person skilled in the art.

In yet another embodiment, the host server 12 may obtain one or more value of biological characteristics in response to a request from the user device 16 by indexing a suitable database 18, such as a database associated with a user account for another software service or application storing value of biological characteristics for the user.

In the present case, having obtained a set of values if one or more of biological characteristics at step 22, the depicted method 21 then determines a basal metabolic rate estimation, for the user 20 based on the one or more of the entered or otherwise obtained set of values of biological characteristics.

In certain embodiments, determining the basal metabolic rate estimation for the user 20 involves using one or more mathematical expressions which determine an estimated basal metabolic rate based on the entered or otherwise obtained set of values of biological characteristics. For example, in one embodiment, the basal metabolic rate estimation is obtained using the “Revised Harris Benedict Roza” equation as would be well understood by a person skilled in the art. However, it is possible that other equations or techniques for obtaining a basal metabolic rate estimation from one or more of the user's personal attributes may be used.

In the present case, by application of the “Revised Harris Benedict Roza” equation, a basal metabolic rate estimation (BMR) estimation for a male user may be determined as:

BMR=88.362+(13.397×Wt)+(4.799×Ht)−(5.677×A) Eq. 1

### Where:

Wt is the user's body weight in kg;
Ht is the user's height in cm; and
A is the user's age in years.

Similarly, by application of the “Revised Harris Benedict Roza” equation, a BMR estimation for a female user may be determined as:

BMR=447.893+(9.247×Wt)+(3.098×Ht)−(4.330×A) Eq. 2

Although the above describe examples involve the use of the “Revised Harris Benedict Roza” equation for obtaining a BMR estimation, it is possible that other suitable techniques or equations may be used for obtaining an estimated energy requirement.

One example of another suitable equation for obtaining an estimated energy requirement includes the “Harris Benedict Equations” published in Harris J A, Benedict F G. A biometric study of human basal metabolism. Proc Natl Acad Sci USA 1918; 4(12):370-3.

Yet another example of suitable equations for obtaining an estimated energy requirement includes the IOM Equation-Estimated Energy Requirement (EER) Estimation of total calories needed as published in the “2005 Dietary Guidelines for Americans and the new food pyramid, MyPyramid”.

Still another example of suitable equations for obtaining an estimated energy requirement includes the “Schofield Equations” as published in Schofield WN (1985). “Predicting basal metabolic rate, new standards and review of previous work”. Hum Nutr Clin Nutr. 39 Suppl 1: 5-41.

Other equations and techniques would be well known to a person skilled in the art.

Having determined the BMR estimation, the method 21 then processes, at step 26, the BMR estimation for the user 20, potentially with one or more other user attributes and/or requirements, to select or determine, at step 28, a caloric and/or macronutrient profile for the user 20. In certain embodiments, and as will be described below, determining a caloric and/or macronutrient profile for the user 20 involves indexing one or more other user attributes and/or requirements into a database to retrieve a particular caloric and/or macronutrient profile having a set of caloric and/or macronutrient ranges and/or values, at least some of which depend on the user attributes and/or requirements and the user's BMR.

For example, in embodiments which obtain one or more other user attributes and/or requirements, the user attributes may include an attribute classifying an activity level of the user 20. For example, in one embodiment, the one or more user attributes include an activity level attribute which classifies the user 20 according to an activity scale or rating ranging from sedentary (or inactive) to very active. The scale or rating may include a set of activity level classifications, such as “sedentary”, “lightly active”, “moderately active” and “very active”.

The one or more user attributes and/or requirements may also include an attribute classifying a “goal” of the user 20. For example, in some embodiments, a user 20 may provide a goal attribute which classifies a user's desired goal or objective associated with implementing diet management using an embodiment of the present disclosure. Example goal attributes include “weight loss”, “better health” and “muscle gain”. It will of course be appreciated that other goal attributes maybe used.

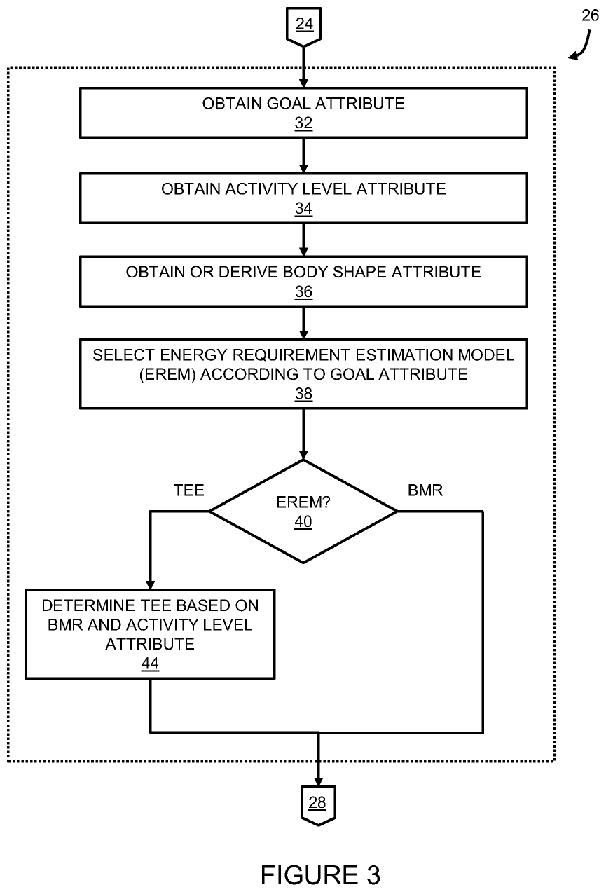

The one or more user attributes and/or requirements may also include an attribute classifying an “activity type” of the user 20. For example, in some embodiments, a user 20 may provide an activity type attribute classifying a predominant type of activity to be conducted as a part of diet management using an embodiment of the present disclosure attribute. Examples of activity type attributes include as “cardio”, “endurance”, “high intensity” and “resistance training”. Turning now to FIG. 3 there is shown a flow diagram of one example for performing step 26 of the method 21 depicted in FIG. 2. As shown, in this example a set of one or more values of biological characteristics for user attributes is obtained, as will be described below.

At step 32, a goal attribute classifying the user's 20 desired goal or objective associated with implementing diet management using an embodiment of the present disclosure is obtained. In the present case, as described above, the goal classification attribute may include one of “weight loss”, “better health” or “muscle gain”. An advantage of obtaining a goal classification attribute is that it may allow for the generation of a caloric or macronutrient profile which takes into account the user's 20 goals.

At step 34, an activity classification attribute is obtained which classifies the according to an activity scale or rating ranging from sedentary (or inactive) to very active. An advantage of obtaining an activity level attribute is that it may allow for an adjustment or correction of the estimated energy requirement obtained at step 24 (ref FIG. 2) according to the activity level of the user 20, depending on the basis for the energy requirement estimation. For example, if the estimated energy requirement is a BMR estimation, and the activity level attribute is “active” or “very active”, the estimated energy requirement may be recalculated as a “total energy expenditure” (TEE) based on the BMR estimation. On the other hand, if the estimated energy requirement is a BMR estimation, and the activity level attribute is “sedentary”, the estimated energy requirement may be recalculated as a “total energy expenditure” (TEE) based on the BMR.

At step 36, in this example, a user attribute is obtained in the form of a somatotype attribute which classifies the user's somatotype as one of an ectomorph, mesomorph or endomorph body type. An advantage of obtaining a somatotype attribute is that it may allow for the generation of a caloric or macronutrient profile which takes into account the user's 20 body shape and related physiological characteristics, including metabolic rate related characteristics.

Having obtained a goal attribute, an activity level attribute and a somatotype attribute, the method then selects, at step 40, an estimated energy requirement model (EREM) depending on the goal classification attribute.

As describe above, in certain embodiments either a TEE based estimation or a BMR based estimation is selected as a final energy requirement estimation depending on user 20 attributes and/or one or more classification attributes.

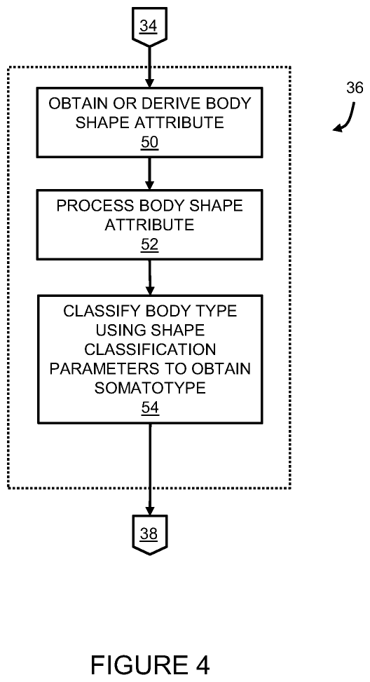

FIG. 4 to FIG. 6 show flow diagrams for different example approaches for obtaining a somatotype attribute classifying the user's 20 somatotype as one of, for example, an ectomorph, mesomorph or endomorph. Before continuing, although the described examples involve classifying the user's 20 somatotype as one of an ectomorph, mesomorph or endomorph body type, it will be appreciated that other types of body classification attributes may be used. For example, in some embodiments the user's 20 body type may be classified as “hourglass”, “inverted triangle”, “triangle”, “rectangle”, or “diamond”. In another embodiment, the user's 20 body type may be classified as “thin-long”, “stout” or “motile”. In yet another embodiment, the user's 20 body type may be classified quantitatively.

Referring now to FIG. 4, in this example approach a user 20 enters body measures into the user device 16 via a suitable user interface. For example, a user 20 may enter measurements for height, shoulder width, waist, hip, high hip, and chest size. Having received the body measures, device 16 may either communicate these parameters to host server 12 to process, at step 52, the parameters, or process these internally, to classify, at step 54 the user's body type with a specific body type classification attribute. Techniques for classifying a body type with a body type classification attribute according to body shape parameters would be known to a skilled person.

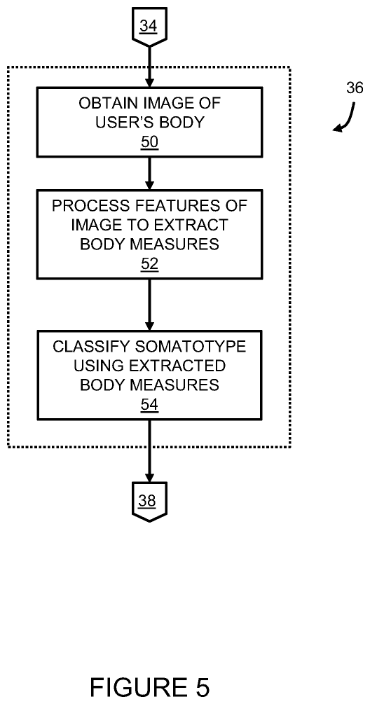

FIG. 5 shows another example technique for obtaining a body classification attribute classifying the user's 20 body type. In this example, a user 20 uses the user device 16 to obtain, at step 50, one or more images of their body. Features of the image are then processed, at step 52, to extract body shape parameters, such as anthropometric measurements, which are then used, at step 54 to classify the user's body type with a specific body type classification attribute. Techniques for classifying a body type with a body type classification attribute according to anthropometric measurements extracted from one or more images of a body would be known to a skilled person. One example of a suitable technique is described in Yang, Jinyan & Li, Yu & Jiang, Tao & Wei, Yu & Xu, Guanlei. (2013). Body Shape Analysis via Image Processing. 10.2991/erse.2013.30.

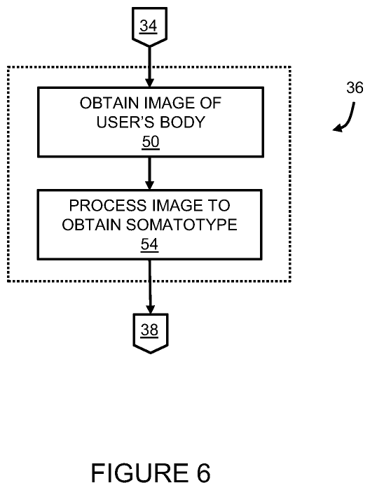

FIG. 6 shows another example technique for obtaining a body classification attribute classifying the user's 20 body type. In this example, a user 20 uses the user device 16 to obtain, at step 50, one or more images of their body. The obtained image is then processed, at step 52, to extract one or more a silhouettes or outlines of the user's body shape which are then used, at step 54 to classify the image and thereby obtain a user's body type with a specific body type classification attribute. Techniques for classifying an image to obtain a body type classification from a silhouette or outline would be known to a skilled person. One example of a suitable technique is described in Dibra, Endri & Jain, Himanshu & Oztireli, Cengiz & Ziegler, Remo & Gross, Markus. (2016). HS-Nets: Estimating Human Body Shape from Silhouettes with Convolutional Neural Networks. 108-117. 10.1109/3DV.2016.19.

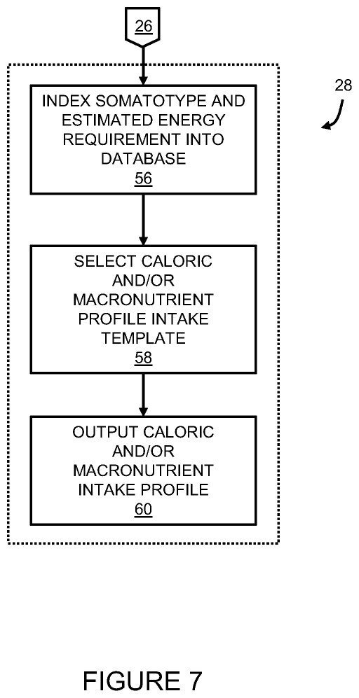

Turning now to FIG. 7 there is shown a flow diagram of an example technique for performing step 28 of the method 21 depicted in FIG. 2. As shown, in this example a body shape classification attribute derived from the user's body shape information and a selected energy requirement estimation is indexed, at step 56, into database 18, to retrieve, at step 58 a caloric and/or macronutrient profile template. An individualised caloric and/or macronutrient intake profile is then selected, at step 60, for the user 20.

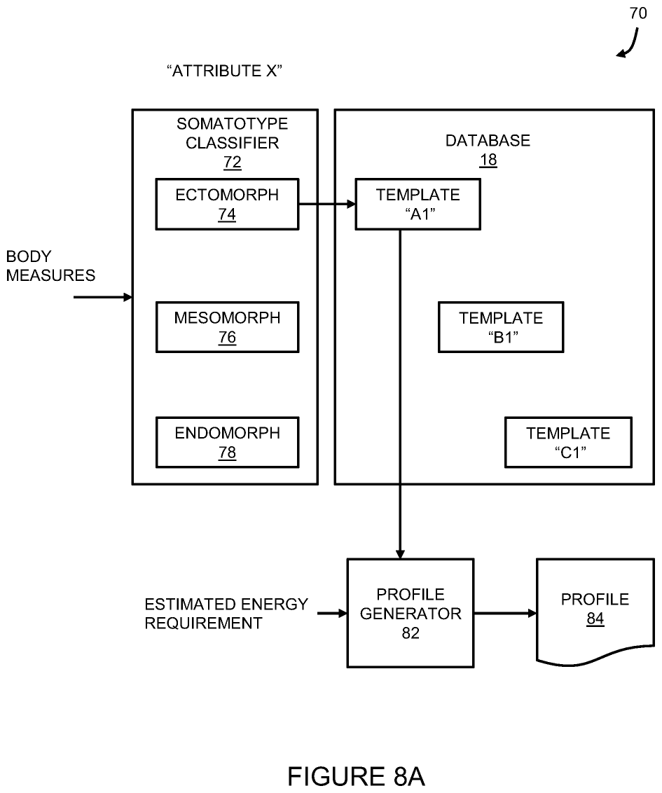

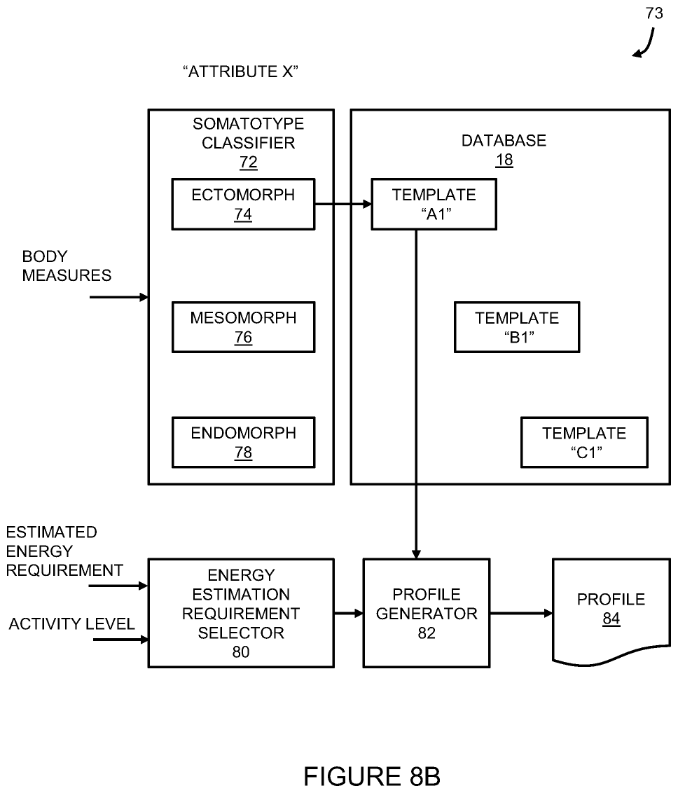

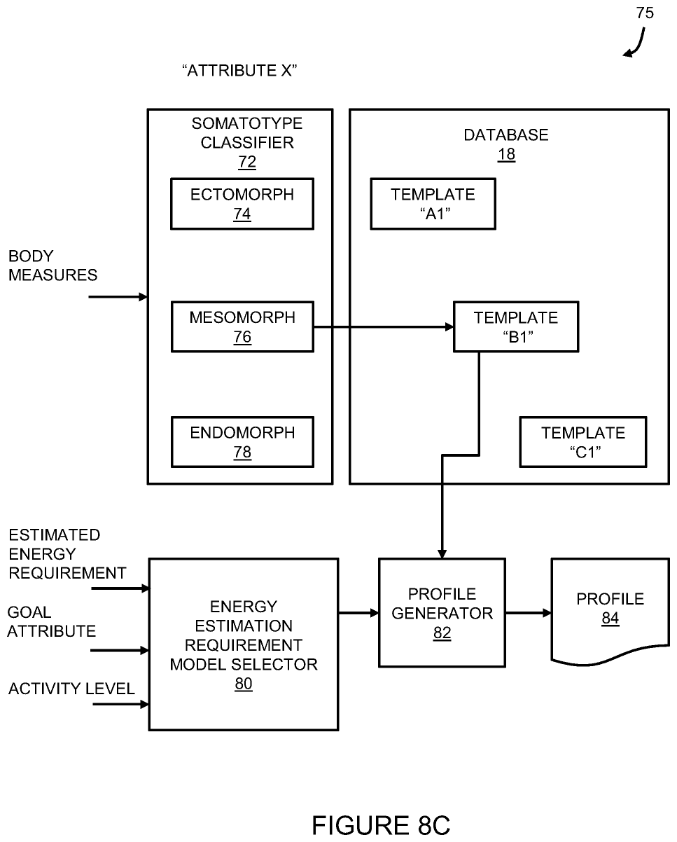

With reference now to FIG. 8A, there is shown an example functional block diagram 70 including functional blocks for performing steps 26 and 28 (ref. FIG. 2) of the method 21 depicted in FIG. 2 and which thus results in providing a caloric and/or macronutrient profile depending on the estimated energy requirement (resulting in selection 80) and, in this example, body shape information for the user.

The illustrated functional block diagram includes a body shape classifier 72, profile generator 82, and the database 18. Database 18 includes plural caloric and/or macronutrient intake profile templates, shown here as “TEMPLATE A1”, “TEMPLATE B1” and “TEMPLATE C1”. Each template may include a table of entries of, for fields for, respective caloric parameters and/or macronutrient parameters, at least some of which vary according to the body shape classification attribute with which the template relates. So, for example, “TEMPLATE A1” may comprise a table containing fields for an ectomorph classification. “TEMPLATE B1” may comprise a table containing fields for a mesomorph classification. TEMPLATE C1 may comprise a table containing fields for an endomorph classification.

In the present case, body shape classifier 72 receives body shape information from the user 20. As explained above, the body shape information may comprise one or more images of the user's body, or it may comprise a body type selection entered into the user device by the user 20. Having received the body shape information, the body shape classifier 72 then assigns a selected body shape classification attribute depending on the body shape information. In the present case, the body shape classification attribute is selected as either an ectomorph 74, mesomorph 76, or endomorph 78 classification. Example techniques for assigning a selected body shape classification attribute depending on the body shape information have been described earlier.

Once a body shape classification attribute has been assigned to a user 20, the body shape classification attribute is then indexed into database 18 to retrieve a particular caloric and/or macronutrient intake profile template for the body shape classification attribute. In the illustrated example, the assigned body shape classification attribute is ectomorph 74 classification which indexes into database 18 to retrieve TEMPLATE A1.

Having retrieved a particular caloric and/or macronutrient intake profile template, profile generator 82 then populates the particular caloric and/or macronutrient intake profile template with caloric and/or macronutrient values, or ranges of such values, depending on the estimated energy requirement to provide an individualised caloric and/or macronutrient intake profile 84 for the user 20. In this example, the energy estimation requirement is provided as, for example, the user's BMR or TEE value or range of values and the retrieved caloric and/or macronutrient intake profile template is populated according to the value or range of values.

Turning now to FIG. 8B, there is shown another example functional block diagram 73 including functional blocks for performing steps 26 and 28 (ref. FIG. 2) of the method 21 depicted in FIG. 2 and which thus results in determining a caloric and/or macronutrient profile depending on the processed estimated energy requirement (resulting in selection 80) at and body shape information for the user.

The illustrated functional block diagram includes a body shape classifier 72, energy estimation requirement model selector 80, profile generator 82, and the database 18. Database 18 includes plural caloric and/or macronutrient intake profile templates, shown here as “TEMPLATE A1”, “TEMPLATE B1” and “TEMPLATE C1”.

In the present case, body shape classifier 72 receives body shape information from the user 20. As explained above, the body shape information may comprise one or more images of the user's body, or it may comprise numerical information (such as size information) entered into at the user device by the user 20, or may comprise a body type selection entered into the user device by the user 20. Having received the body shape information, the body shape classifier 72 then assigns a selected body shape classification attribute depending on the body shape information. In the present case, the body shape classification attribute is selected as either an ectomorph 74, mesomorph 76, or endomorph 78 classification. Example techniques for assigning a selected body shape classification attribute depending on the body shape information have been described earlier.

Once a body shape classification attribute has been assigned to a user 20, the body shape classification attribute is then indexed into database 18 to retrieve a particular caloric and/or macronutrient intake profile template for the body shape classification attribute. In the illustrated example, the assigned body shape classification attribute is an ectomorph 74 classification which is indexed into database 18 to retrieve TEMPLATE A1.

Having retrieved a particular caloric and/or macronutrient intake profile template, profile generator 82 then populates the particular caloric and/or macronutrient intake profile template with caloric and/or macronutrient values, or ranges of such values, including values which depend on the estimated energy requirement and the activity level attribute, to provide an individualised caloric and/or macronutrient intake profile 84 for the user 20. In this example, the energy estimation requirement selector selects either the BMR or a TEE as the estimated energy requirement depending on the goal attribute. For example, for a goal attribute of “Fat Loss” the user's BMR may be used as the energy estimation requirement, whereas for goal attributes of “Muscle Gain” or “Better Health” the BMR may be used to derive the estimated energy requirement as the user's TEE.

FIG. 8C shows another functional block diagram 75 similar to that shown in FIG. 8A. However, in the example depicted in FIG. 8B, the body shape classification attribute assigned to the user 20 is a mesomorph 76 classification which is indexed into database 18 to retrieve TEMPLATE B1.

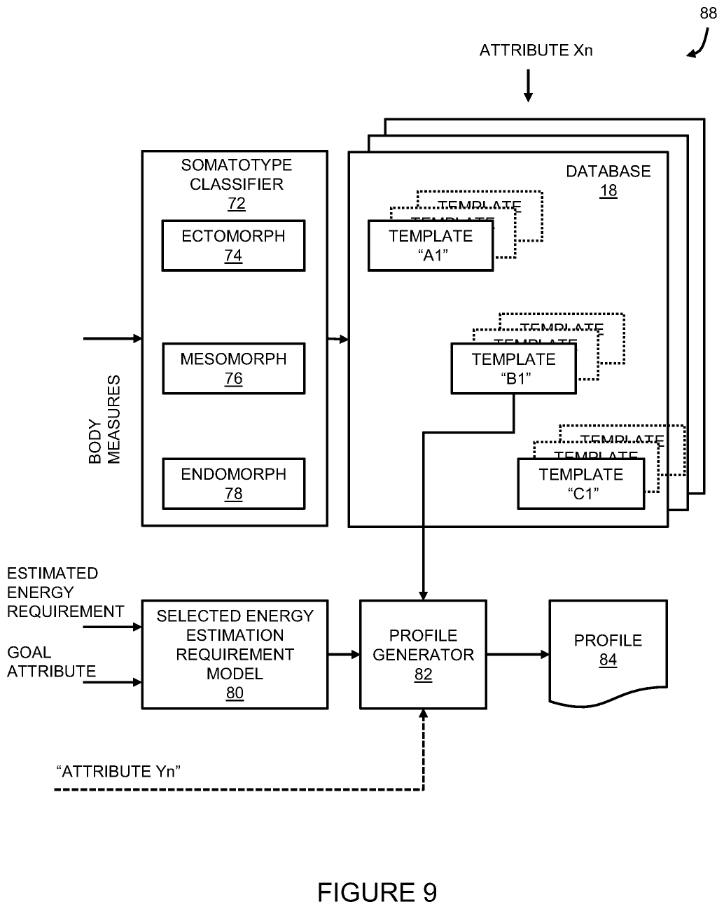

Turning now to FIG. 9 there is shown another example of a functional block diagram 88 including functional blocks for performing steps 26 and 28 (ref FIG. 2) of the method 21 depicted in FIG. 2. In this example, an additional attributes (Xn) is indexed into database 18 with the body shape classification attribute to retrieve a template depending on the body shape classification attribute and the additional attributes (Xn). Thus, in this example, the database effectively stores a 2-dimensional array of templates. In the depicted example, the additional attribute could include, for example, an activity type attribute of the type described previously.

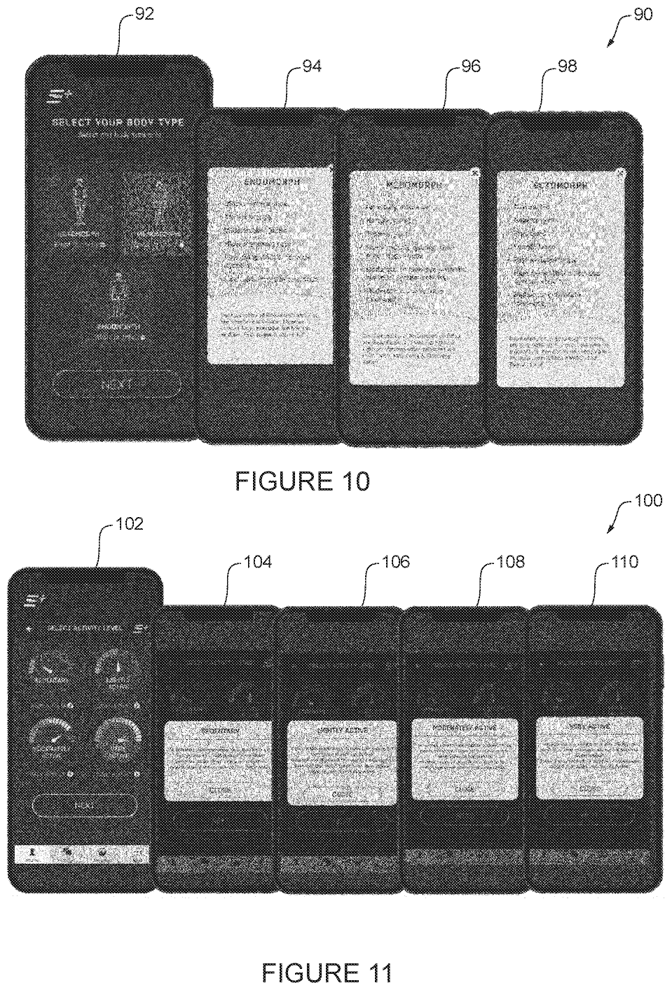

FIGS. 10 to 12 show examples of user interface display screens presented to the user during an embodiment of the disclosure.

Referring to FIG. 10, there is shown a series of display screen interfaces 90 associate with an example approach for obtaining a body shape classification attribute. In this example, a user interacts with user interface display screen 92 to select a representation of a body shape approximating their own body shape. Interface display screens 94, 96, 98 are then shown to the user, depending on the selection, to provide information to assist with the selection process. Having made a selection, the body shape classification attribute is then assigned the somatotype selected by the user.

FIG. 11 shows a series of display screen interfaces 100 for an example approach for obtaining an activity level attribute. In this example, a user interacts with user interface display screen 102 to select an activity level. Interface display screens 104, 106, 108, and 110 are then shown to the user, depending on the selection, to provide information to assist with the selection process. Having made a selection, the selection is then assigned as the activity level attribute for the user.

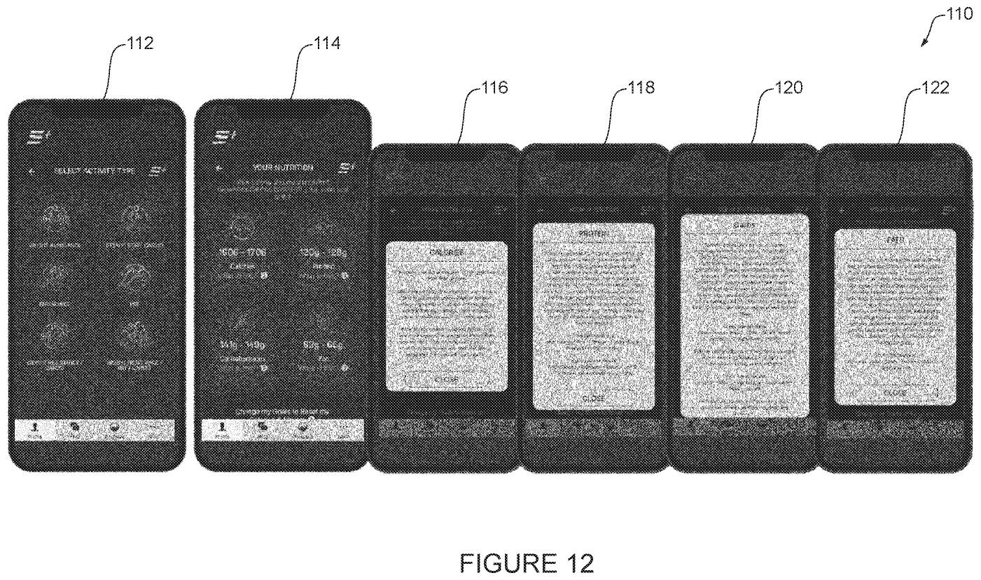

FIG. 12 shows a series of user interface display screens 110 associated with an example approach for obtaining activity level. In this example, a user interacts with user interface display screen 112 to select an activity level representing their intended activity level. Display screens 114, 118, 120, 122 are then shown to the user, depending on the selection, to provide information in relation to the selected caloric and macronutrient profile, which in this example has been selected following the body shape classification attribute selection of FIG. 10, the activity level attribute selection of FIG. 11, and the activity level selection of FIG. 11 together with an estimated energy requirement derived from a basal metabolic rate (BMR) estimation for the user based on the set of values of biological characteristics.

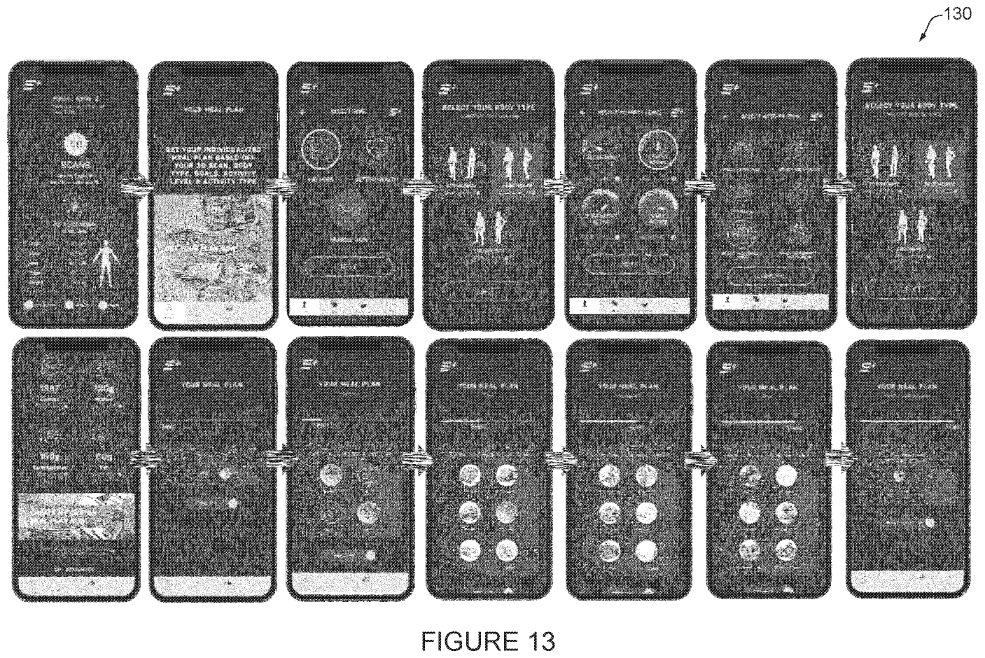

FIG. 13 shows an example of a sequence of display screen interfaces 130 which provides, as an output, a meal plan which has been selected based on a caloric and macronutrient profile which has been selected using an embodiment of the present disclosure. Embodiments of the present disclosure may allow the user to choose from their preferred food choices and automatically be matched to how many meals they wish to eat during the day, ensuring that their total daily counts are the same as the caloric profile.

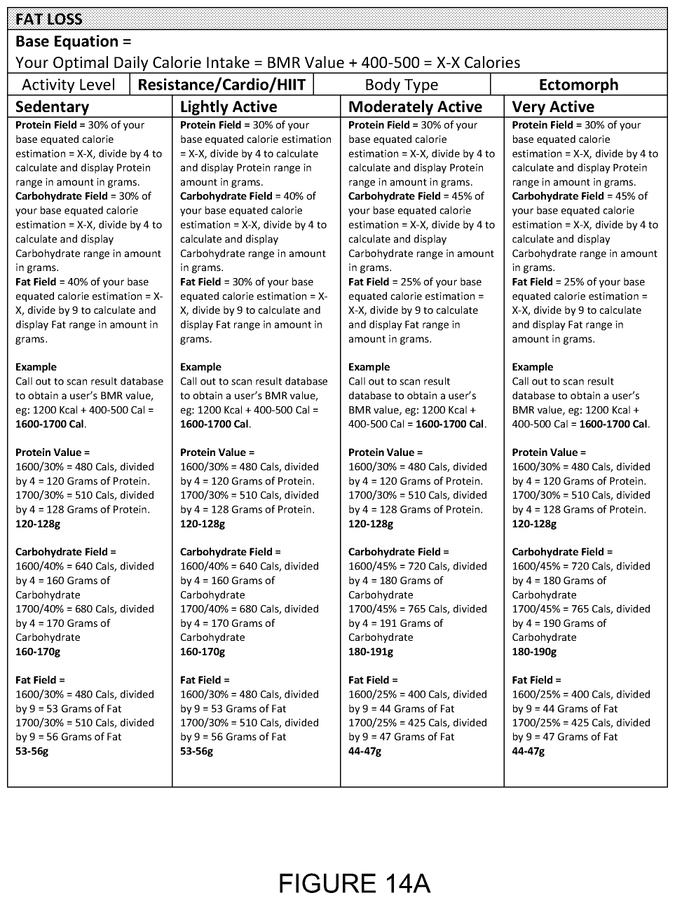

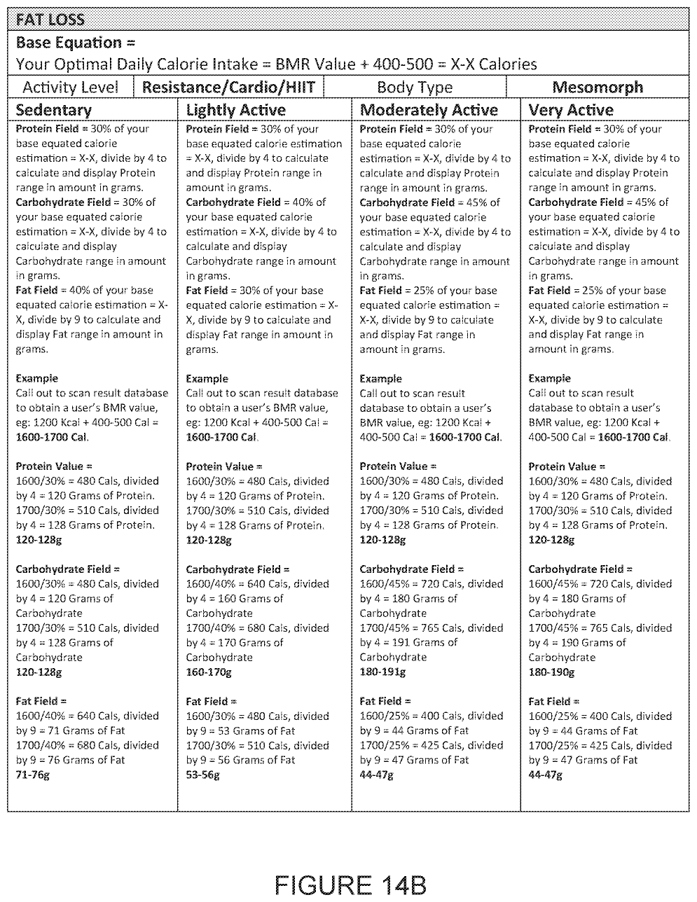

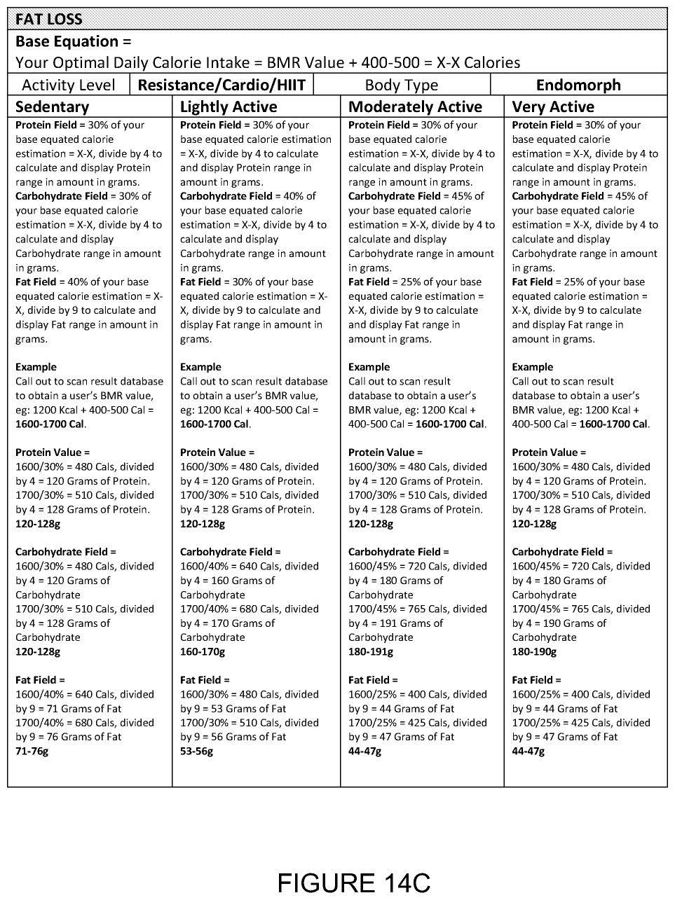

FIGS. 14A to 14C show three examples of a different sets of caloric and macronutrient profile templates for a user having a “Fat Loss” goal attribute and an “Resistance/Cardio/HIIT activity type attribute, with a selected caloric and macronutrient profile depending on the body shape classification attribute and the activity level attribute (eg. “Sedentary”, “Lightly Active”, “Moderately Active”, or “Very Active”).

In the present case, a profile for particular activity level attribute shown in FIG. 14A is determined by populating the relevant fields of a selected template associated with the user's activity level attribute for a user having an “Ectomorph” body shape characteristic, whereas the templates shown in FIG. 14B and FIG. 14C are selectable for a user having an “Mesomorph” and “Endomorph” body shape characteristic attribute respectively.

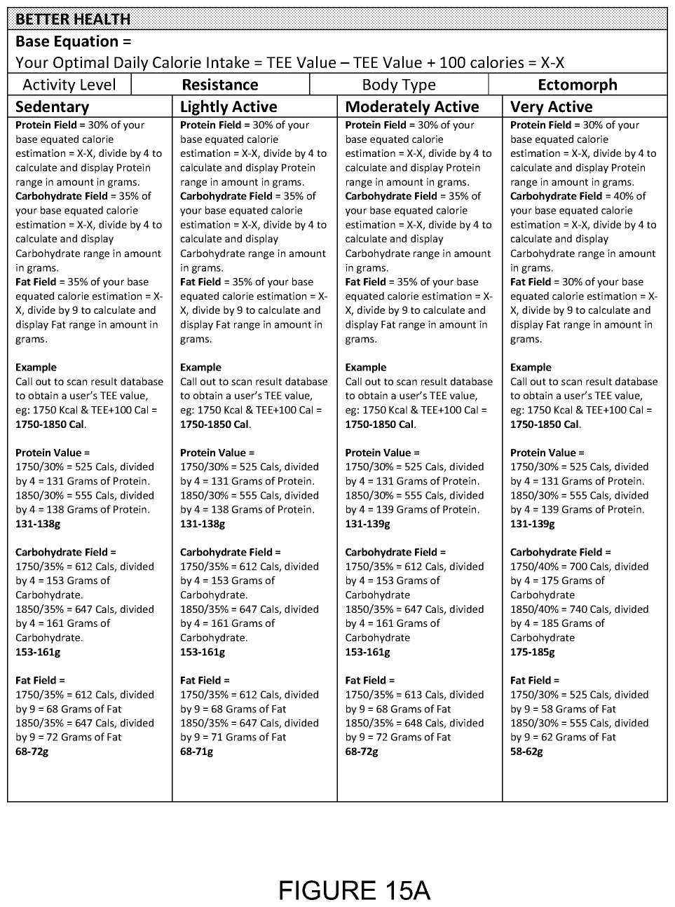

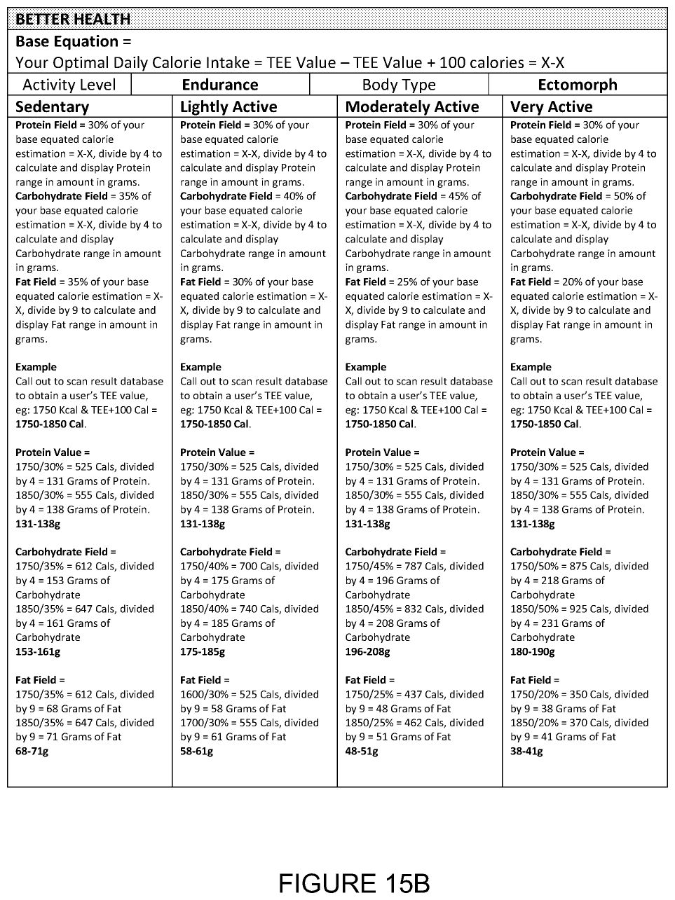

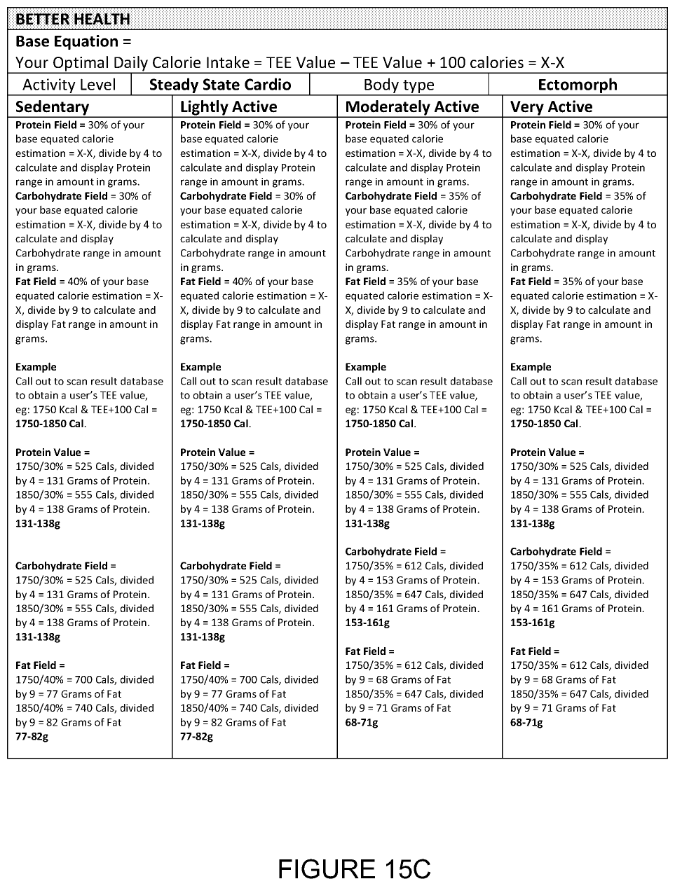

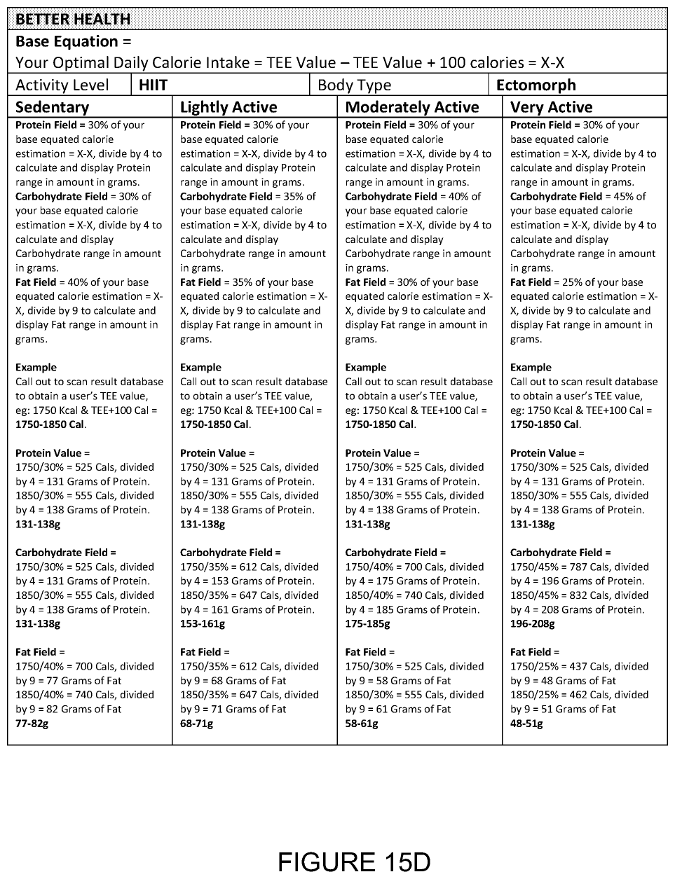

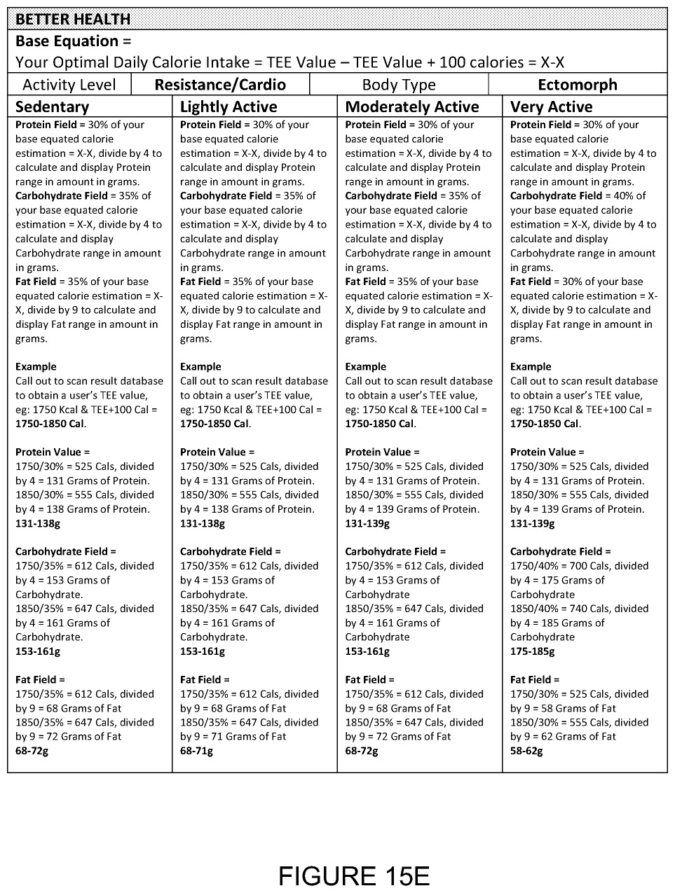

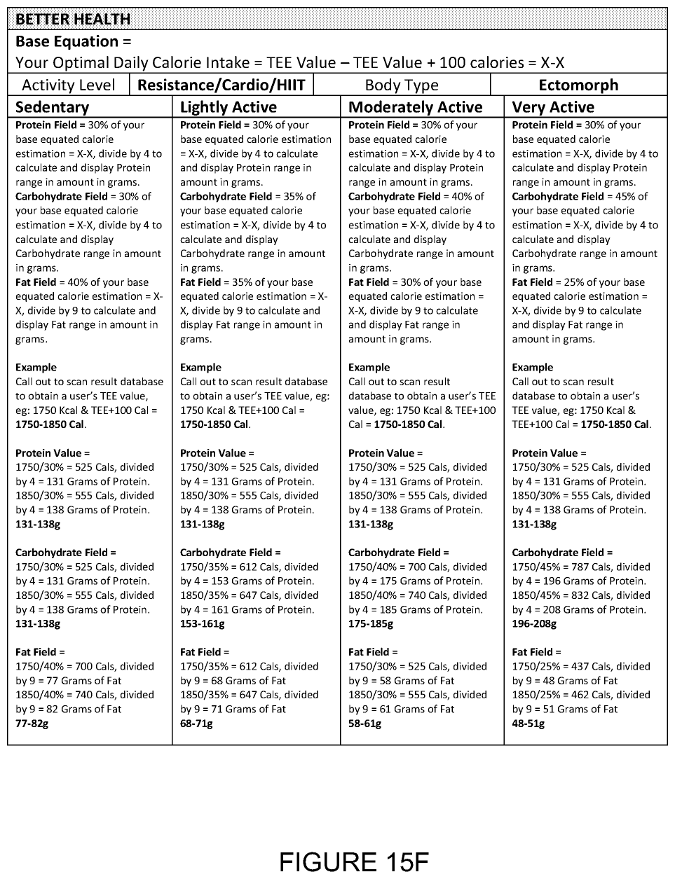

FIGS. 15A to 15F show six examples of a different sets of caloric and macronutrient profile templates for a user having a “Better Health” goal attribute and an “Ectomorph” body shape characteristic attribute, with the selected caloric and macronutrient profile depending on the activity type attribute and the activity level attribute (eg. “Sedentary”, “Lightly Active”, “Moderately Active”, or “Very Active”). In the present case, a profile for particular activity level attribute shown in FIG. 15A is determined by populating the relevant fields of a template selected according to the user's activity level attribute for a user having a “Resistance” activity type attribute, whereas the templates shown in FIG. 15B to FIG. 15F are selectable for a user having an “Endurance”, “Steady State Cardio”, “HIIT”, “Resistance/Cardio”, or “Resistance/Cardio/HIIT” activity level attribute respectively.

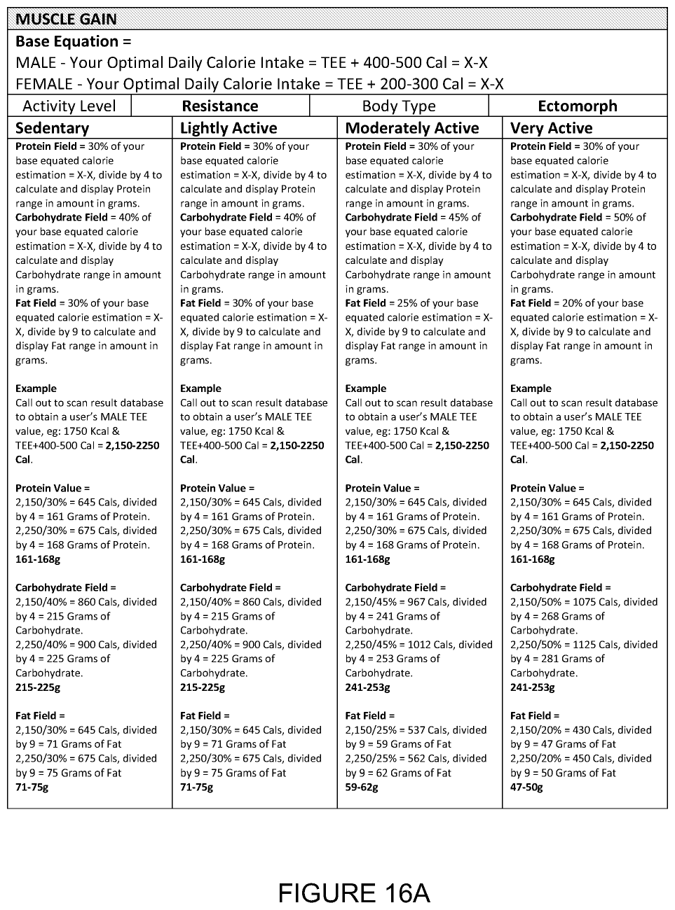

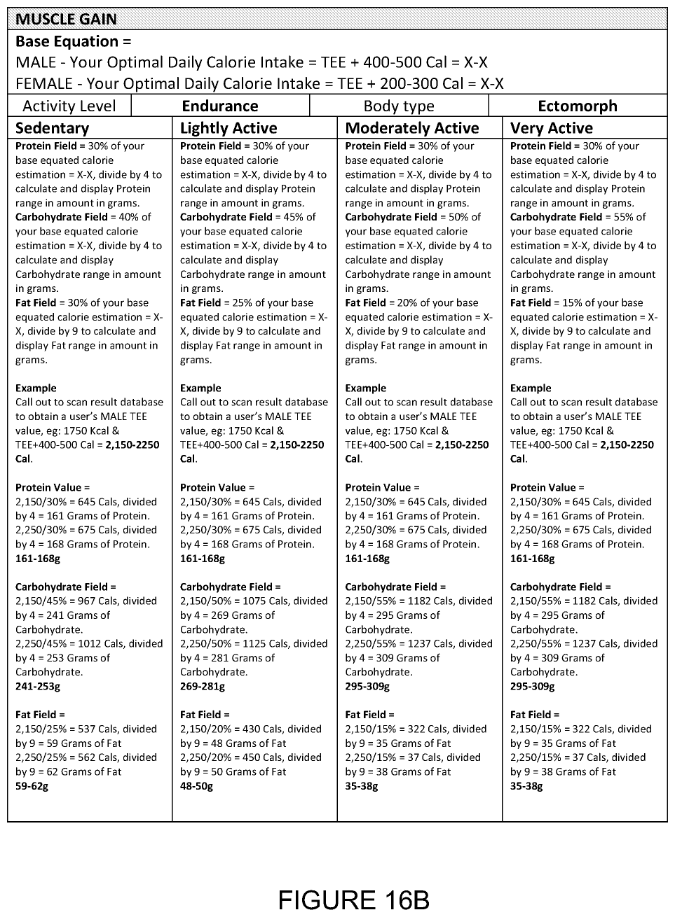

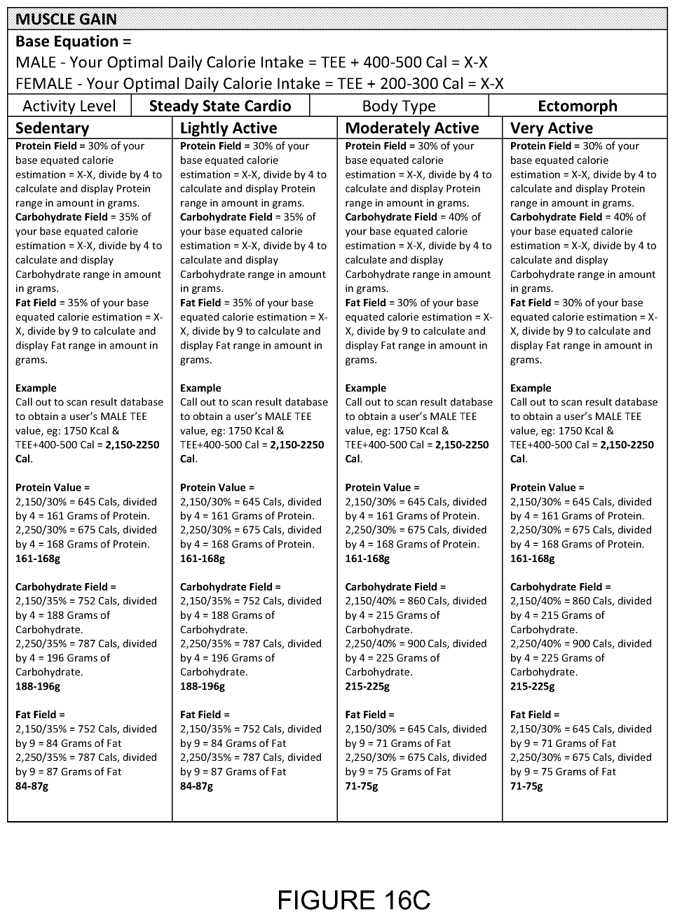

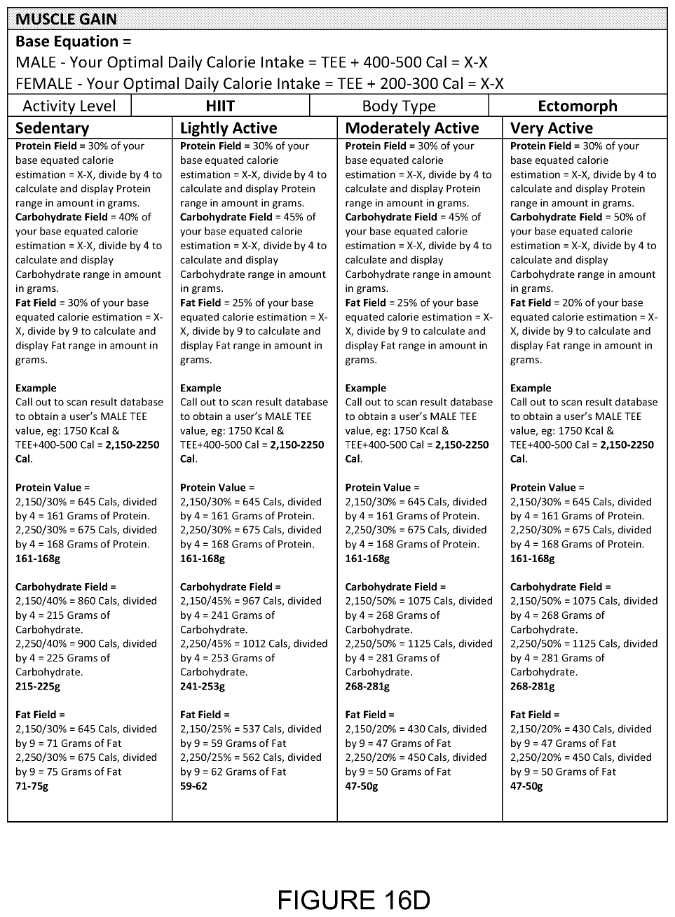

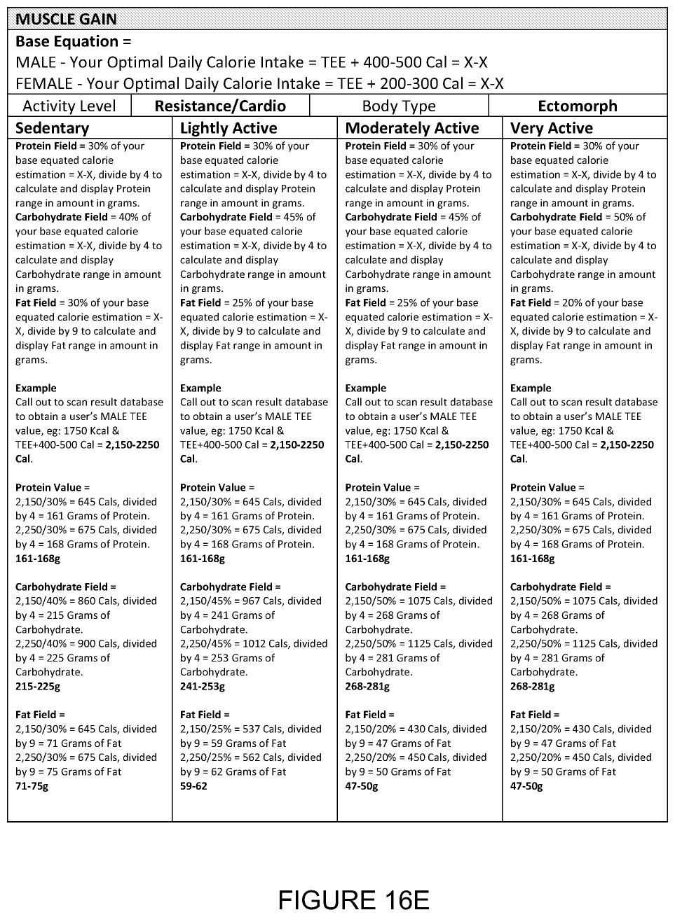

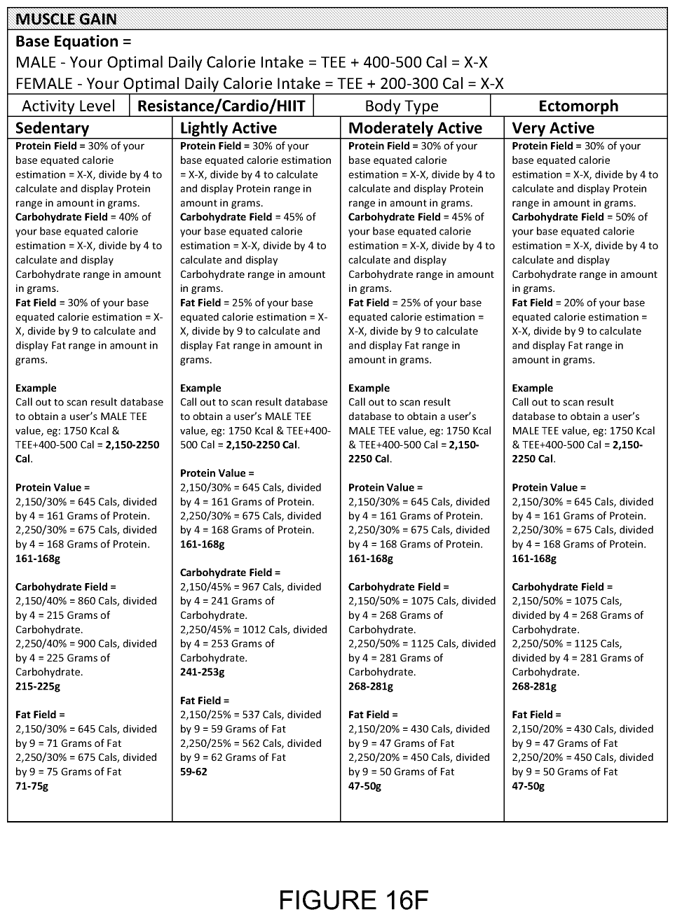

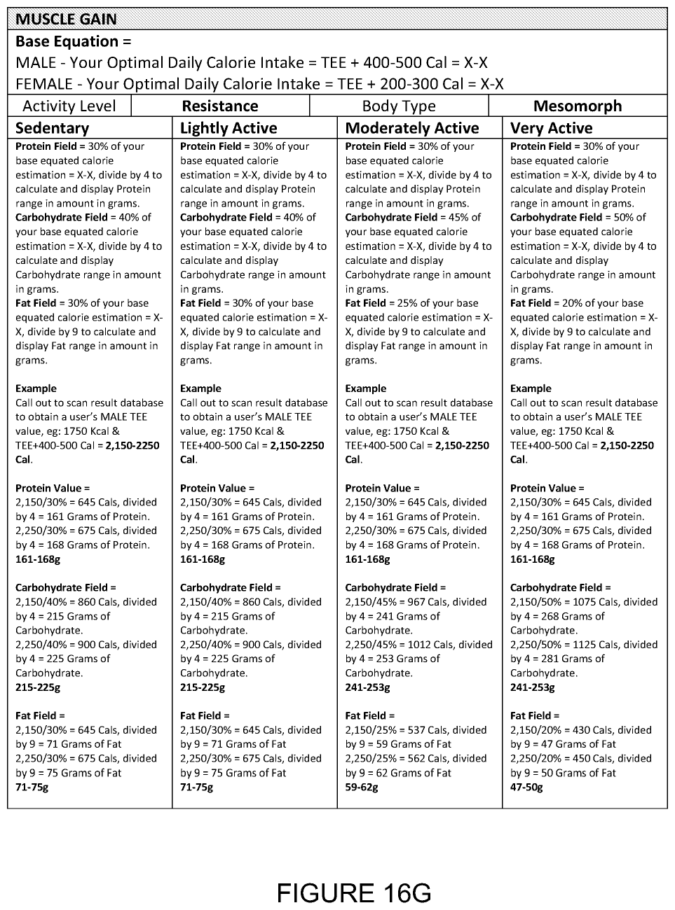

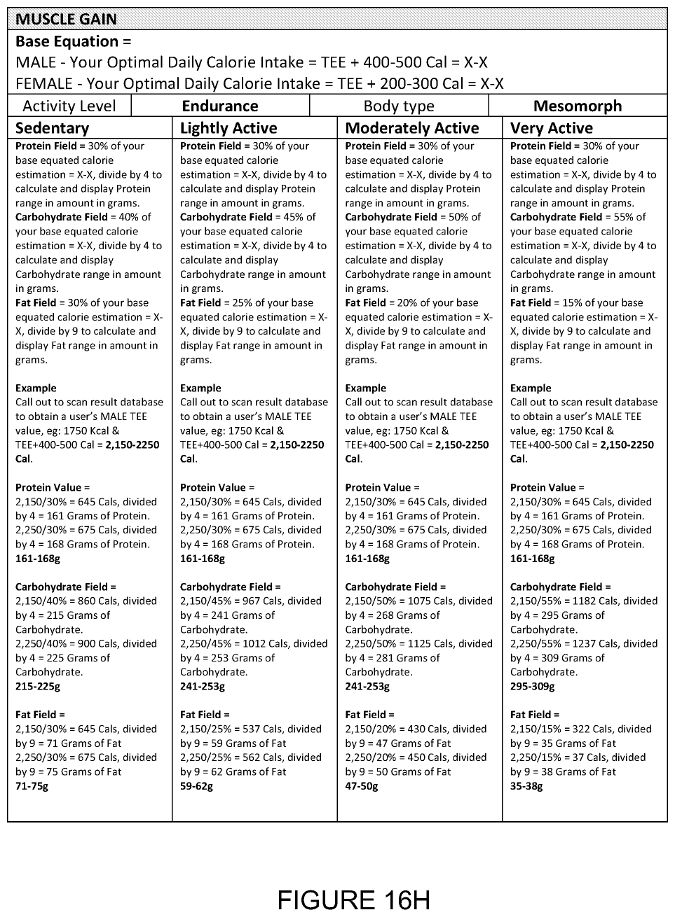

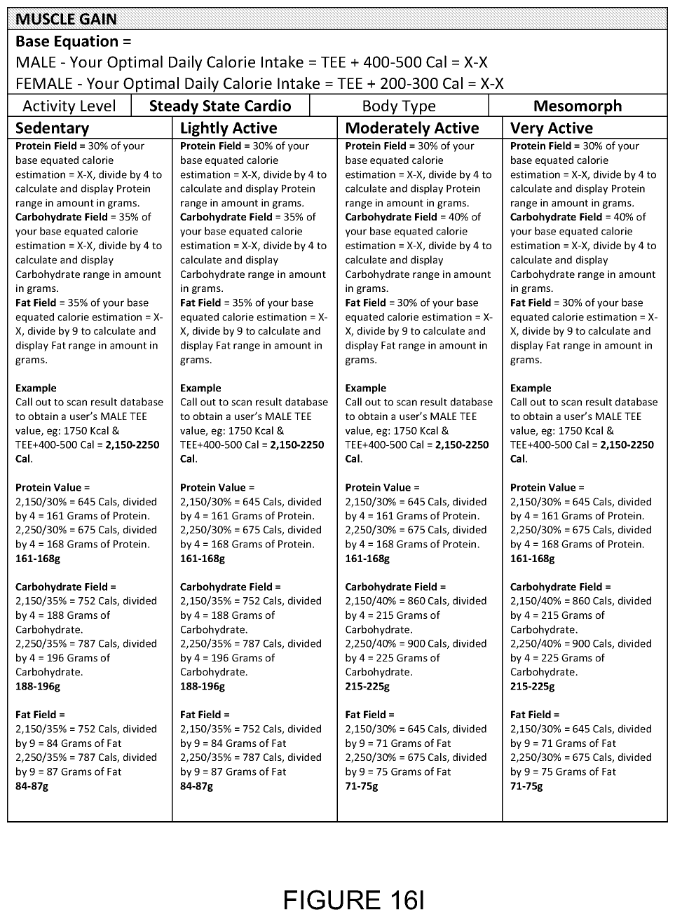

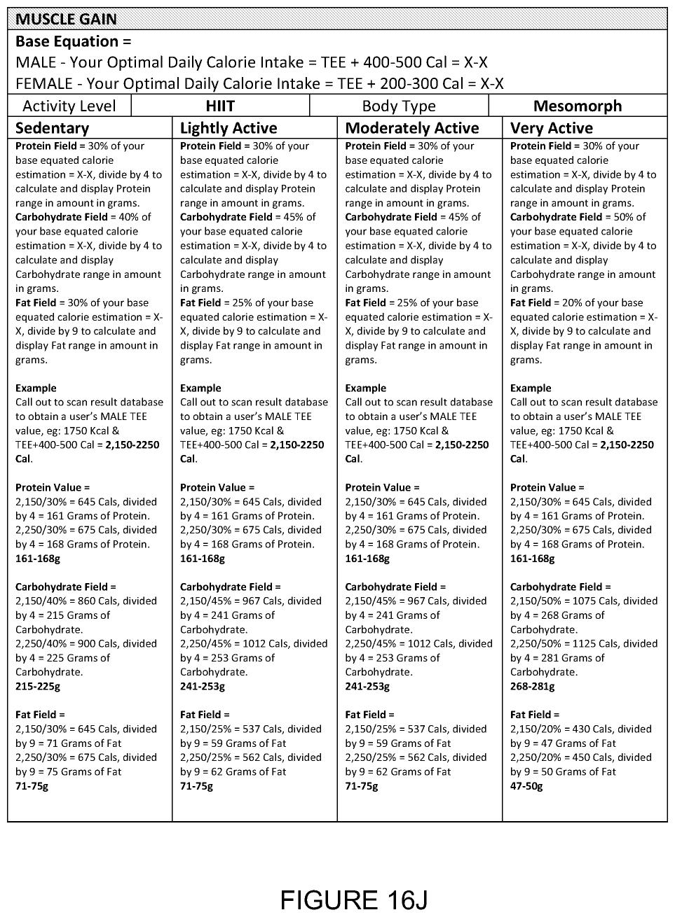

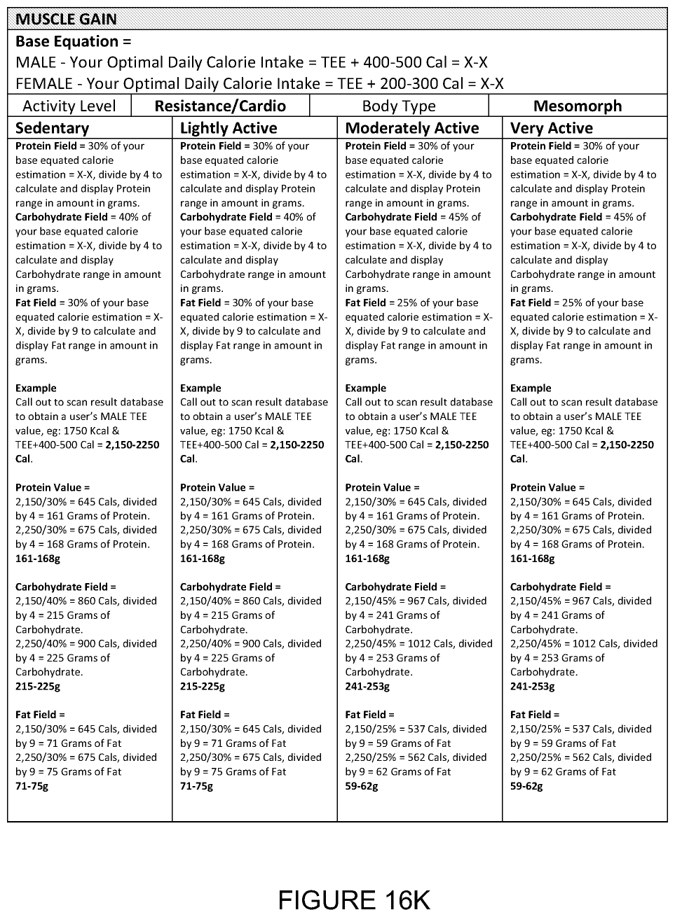

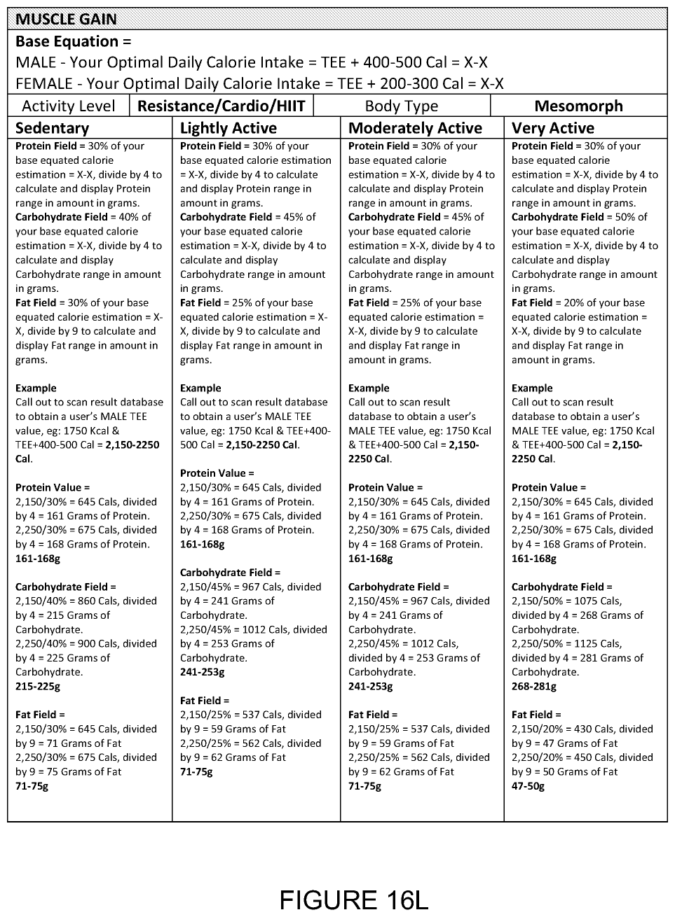

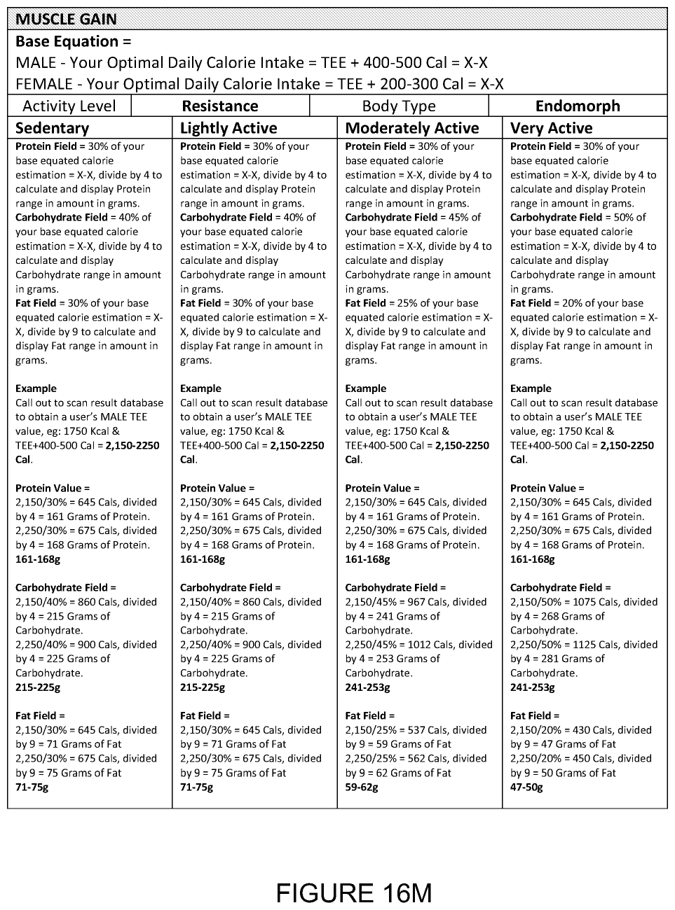

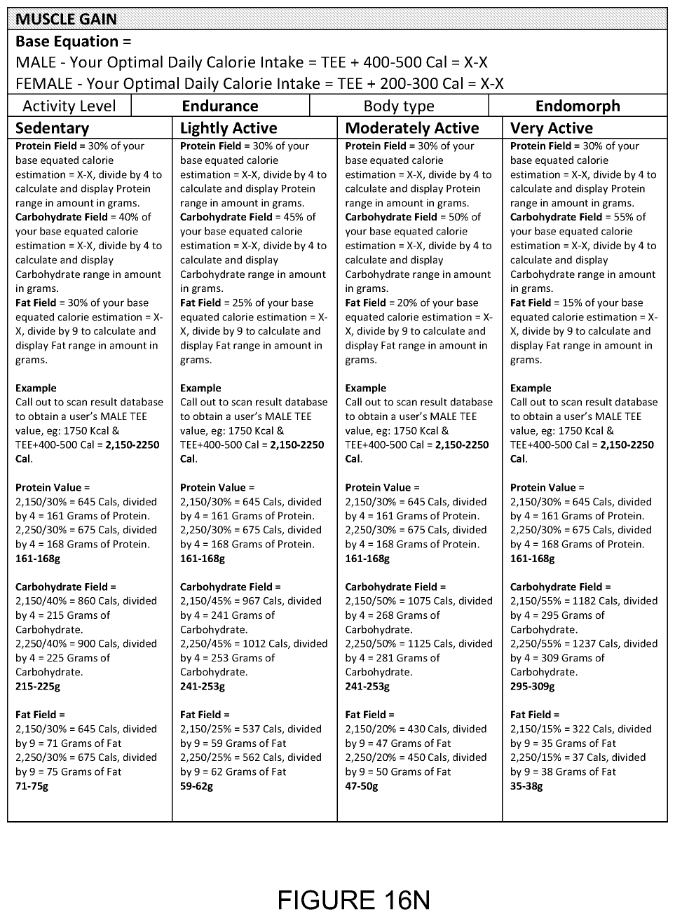

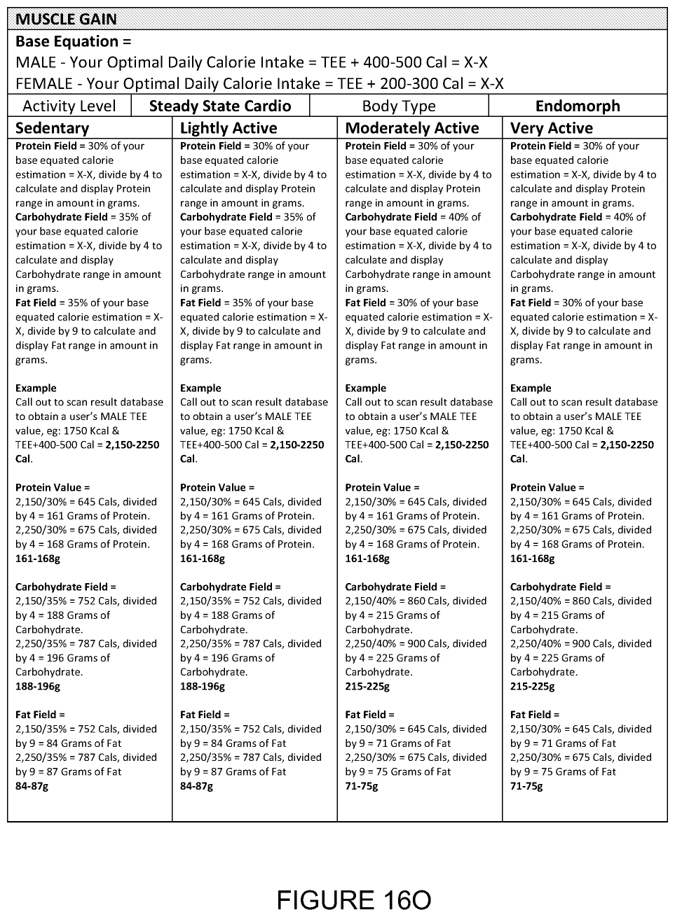

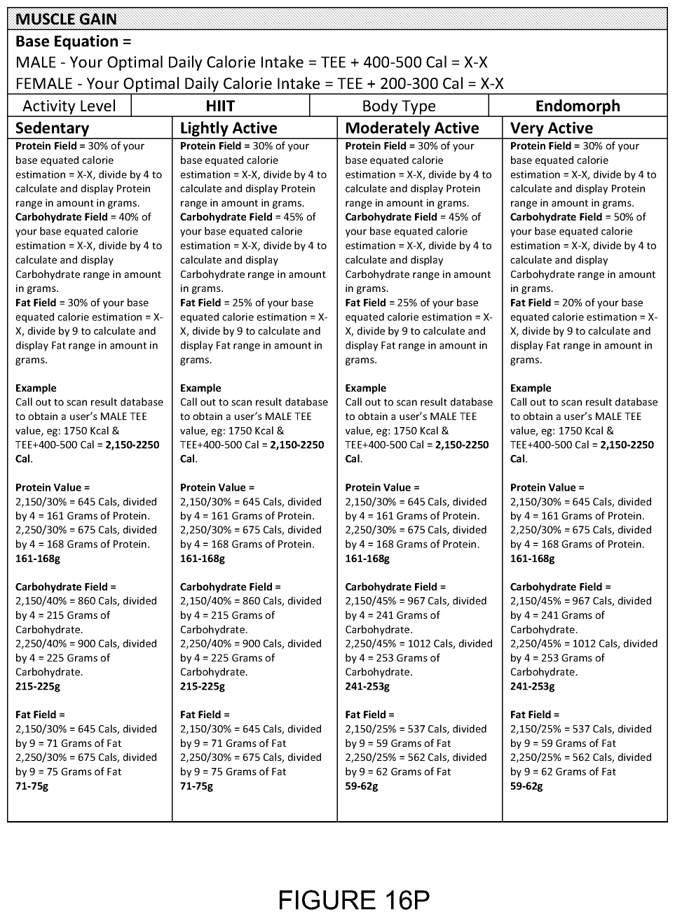

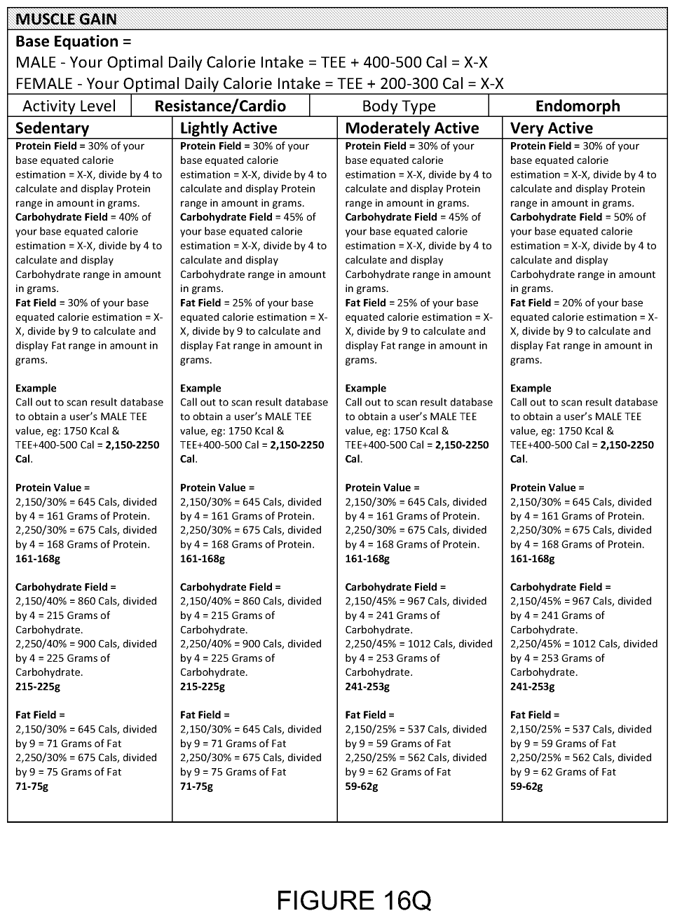

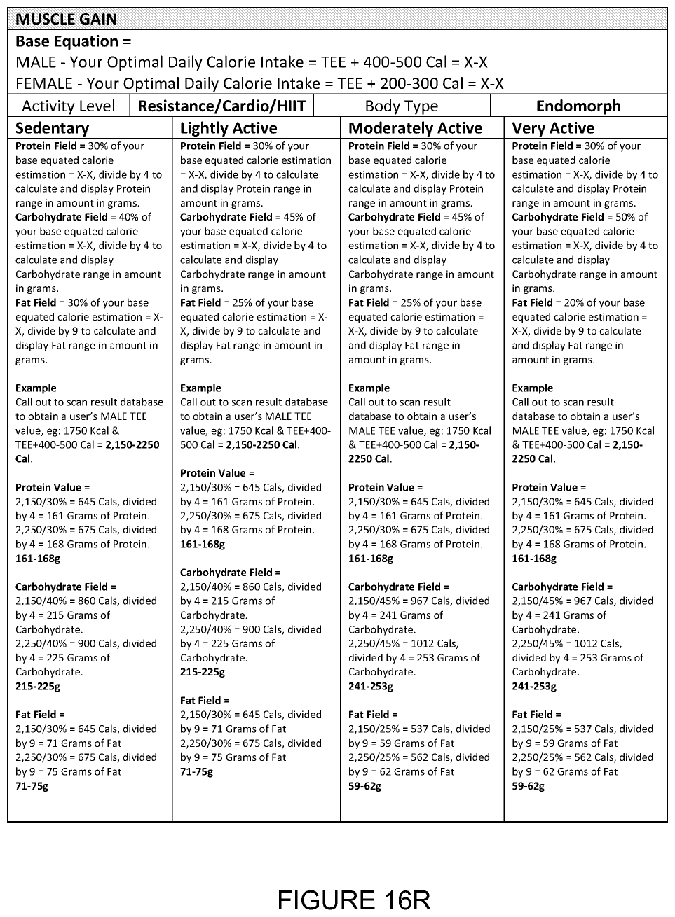

FIGS. 16A to 16R show seventeen template examples of a complete set of caloric and macronutrient profile templates for a user having a “Muscle Gain” goal attribute.

In certain embodiments, populating a template may involve applying adjustments to the caloric and macronutrient values included in the template according to user risk factors and/or an overall health score or index.

For example, in some embodiments, a user's “obesity risk rating” may be assessed based on a sensed or entered waist circumference measurement and assigned, for example, a “Low”, “Moderate” and “High” obesity risk rating attribute based on their waist circumference measurement.

For example, if a user is determined to have a “High” or “Moderate” obesity risk rating, caloric and macronutrient values determined from a template may be adjusted to take into account the risk. For example, for a female having a “High” obesity risk rating, calorie amounts determined using a template may be reduced by 100 calories, whereas for a female having a “Moderate” obesity risk rating, calorie amounts determined using a template may be reduced by 50 calories, for example. Table 1 shows an example approach for assigning an obesity risk rating to a user based on their waist circumference.

TABLE 1

| | | Waist circumference^{b} | |
|---|---|---|---|
| Men | <94 cm | 94-101.9 cm | ≥102 cm |
| Women | <80 cm | 80-87.9 cm | ≥88 cm |
| Classification | Normal fat | Moderate central | High central |
| | distribution | fat accumulation | fat accumulation |
| Risk of | Low | Increased | High |
| co-morbidities | | | |

In certain embodiments, a user may be assigned a value depending on lean body mass versus total fat mass based on the entered and/or sensed values of biological characteristics, such as the user's height, weight, age, gender and waist circumference. In such embodiments, the value may be used to profile a quantitative metric of the user's wellness as compared to a population. The metric may be used to make adjustments to the caloric and macronutrient values included in the template based on the metric.

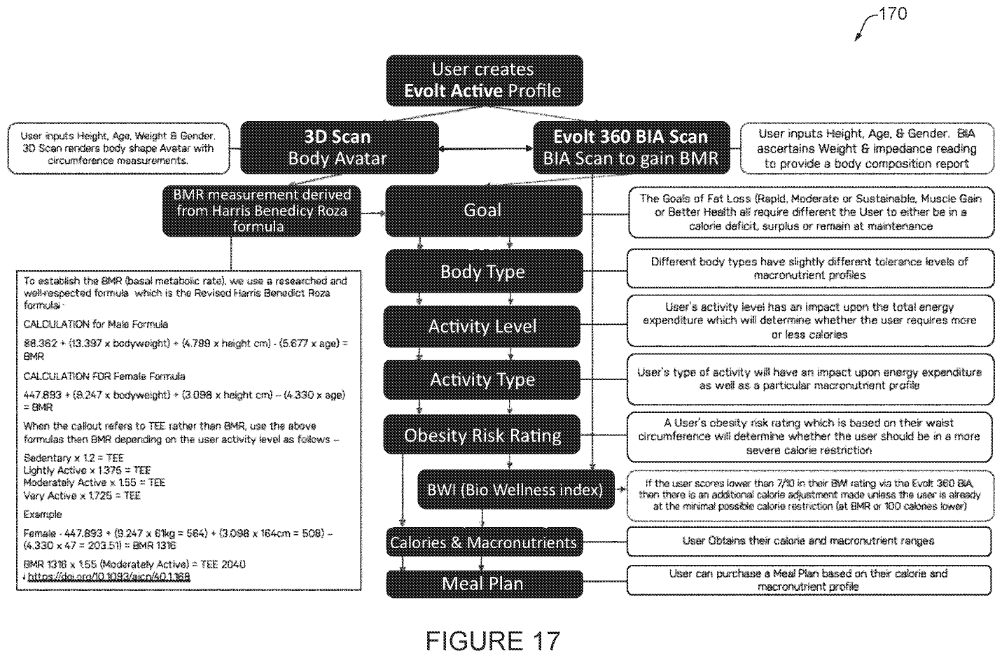

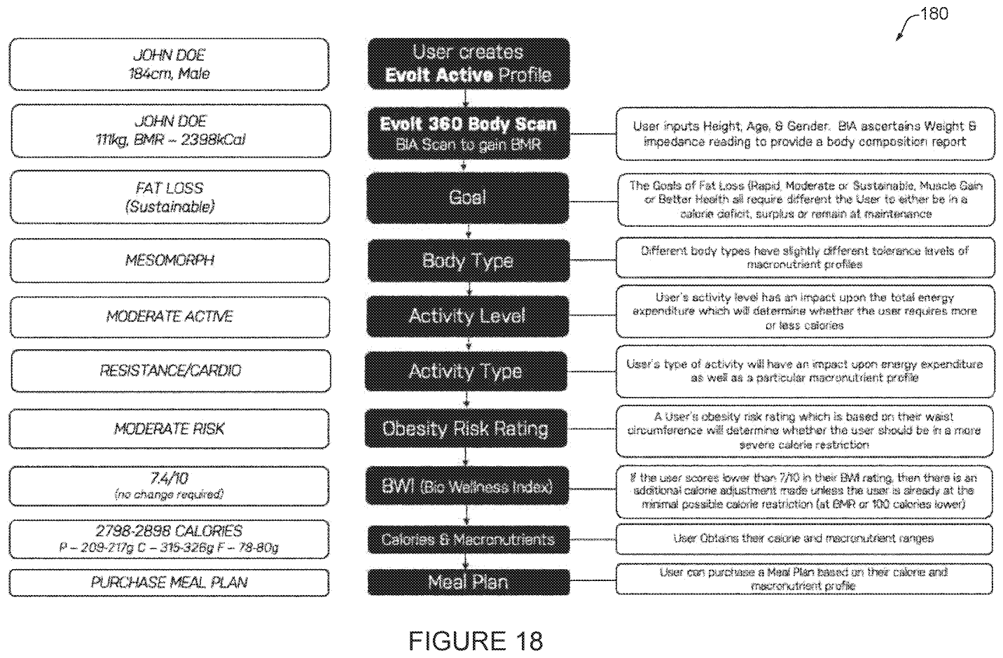

FIGS. 17 and 18, there is show example flow diagrams 170, 180 example processes according to embodiments of the disclosure.

Referring now to the system may be a computer implemented system comprising of a display device, a processor and a memory and an input device. The memory may comprise instructions to cause the processor to execute a method described herein. The processor memory and display device may be included in a standard computing device, such as a desktop computer, a portable computing device such as a laptop computer or tablet, or they may be included in a customised device or system. The computing device may be a unitary computing or programmable device, or a distributed device comprising several components operatively (or functionally) connected via wired or wireless connections.

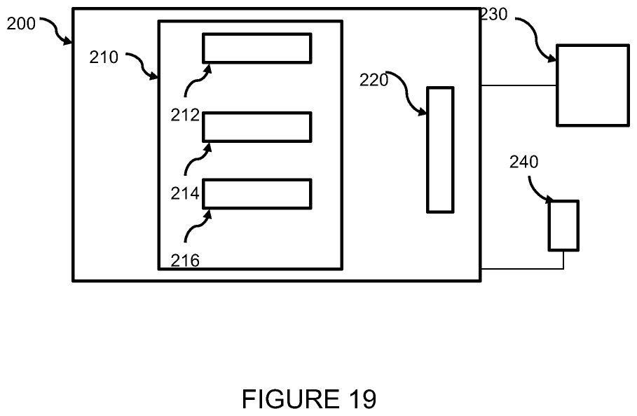

An embodiment of a computing device 200 is illustrated in FIG. 19 and comprises a central processing unit (CPU) 210, a memory 220, a display apparatus 230, and may include an input device 240 such as keyboard, mouse, etc, network communications interface 212 and may include an input device, such as keyboard, mouse, etc. A graphical processing unit (GPU) may also be included.

The central processing unit 210 may comprise a parallel processor, a vector processor, or a distributed computing device. The memory 220 is operatively coupled to the processor(s) and may comprise RAM and ROM components, and may be provided within or external to the system 200. The memory 220 may be used to store the operating system and additional software modules or instructions. The processor(s) may be configured to load and executed the software modules or instructions stored in the memory.

An input/output interface 212 may comprise a network interface and/or communications module for communicating with an equivalent communications module in another device using a predefined communications protocol (e.g. Bluetooth, Zigbee, IEEE 802.15, IEEE 802.11, TCP/IP, UDP, etc).

The display apparatus 230 may comprise a flat screen display (eg LCD, LED, plasma, touch screen, etc), a projector, CRT, etc.

The CPU 210 comprises an input/output Interface 212, an Arithmetic and Logic Unit (ALU) 214 and a Control Unit and Program Counter element 216 which is in communication with input and output devices (e.g. input device 240 and display apparatus 230) through the input/output Interface 212.

In one form, the disclosure may comprise a computer program product, such as software application, including instructions which are executable by a processor to perform the above described method or operations presented herein.

For example, such a computer program product may comprise a computer (or processor) readable medium having instructions stored (and/or encoded) thereon, the instructions being executable by one or more processors to perform the operations described herein.

The processor memory and display device may be included in a standard computing device, such as a desktop computer, a portable computing device such as a laptop computer or tablet, or they may be included in a customised device or system. The computing device may be a unitary computing or programmable device, or a distributed device comprising several components operatively (or functionally) connected via wired or wireless connections.

Those of skill in the art would understand that information and signals may be represented using any of a variety of technologies and techniques. For example, data, instructions, commands, information, signals, bits, symbols, and chips may be referenced throughout the above description may be represented by voltages, currents, electromagnetic waves, magnetic fields or particles, optical fields or particles, or any combination thereof.

Those of skill in the art would further appreciate that the various illustrative logical blocks, modules, circuits, and algorithm steps described in connection with the embodiments disclosed herein may be implemented as electronic hardware, computer software or instructions, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

The steps of a method or algorithm described in connection with the embodiments disclosed herein may be embodied directly in hardware, in a software module executed by a processor, or in a combination of the two. For a hardware implementation, processing may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described herein, or a combination thereof. Software modules, also known as computer programs, computer codes, or instructions, may contain a number a number of source code or object code segments or instructions, and may reside in any computer readable medium such as a RAM memory, flash memory, ROM memory, EPROM memory, registers, hard disk, a removable disk, a CD-ROM, a DVD-ROM, a Blu-ray disc, or any other form of computer readable medium. In some aspects the computer-readable media may comprise non-transitory computer-readable media (e.g., tangible media). In addition, for other aspects computer-readable media may comprise transitory computer-readable media (e.g., a signal). Combinations of the above should also be included within the scope of computer-readable media. In another aspect, the computer readable medium may be integral to the processor. The processor and the computer readable medium may reside in an ASIC or related device. The software codes may be stored in a memory unit and the processor may be configured to execute them. The memory unit may be implemented within the processor or external to the processor, in which case it can be communicatively coupled to the processor via various means as is known in the art.

Further, it should be appreciated that modules and/or other appropriate means for performing the methods and techniques described herein can be downloaded and/or otherwise obtained by computing device. For example, such a device can be coupled to a server to facilitate the transfer of means for performing the methods described herein. Alternatively, various methods described herein can be provided via storage means (e.g., RAM, ROM, a physical storage medium such as a compact disc (CD) or floppy disk, etc.), such that a computing device can obtain the various methods upon coupling or providing the storage means to the device. Moreover, any other suitable technique for providing the methods and techniques described herein to a device can be utilized.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The reference to any prior art in this specification is not, and should not be taken as, an acknowledgement of any form of suggestion that such prior art forms part of the common general knowledge.

It will be appreciated by those skilled in the art that the invention is not restricted in its use to the particular application described. Neither is the present invention restricted in its preferred embodiment with regard to the particular elements and/or features described or depicted herein. It will be appreciated that the invention is not limited to the embodiment or embodiments disclosed, but is capable of numerous rearrangements, modifications and substitutions without departing from the scope of the invention as set forth and defined by the following claims.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

It will be appreciated by those skilled in the art that the invention is not restricted in its use to the particular application described. Neither is the present invention restricted in its preferred embodiment with regard to the particular elements and/or features described or depicted herein. It will be appreciated that the invention is not limited to the embodiment or embodiments disclosed, but is capable of numerous rearrangements, modifications and substitutions without departing from the scope of the invention as set forth and defined by the following claims.

1. A method for providing a personalised caloric and/or macronutrient intake profile for a user, comprising:
obtaining a set of values of biological characteristics for the user, wherein the set includes at least one value derived from sensed information, wherein the at least one value includes a value of an anthropometric measurement and/or a value of a bio-impedance measurement;
determining a basal metabolic rate (BMR) estimation for the user based on the set of values of biological characteristics;
determining an energy requirement estimation based on at least the basal metabolic rate (BMR) estimation; and
determining the personalised caloric and/or macronutrient intake profile for the user based on at least the energy requirement estimation.

2. A method according to claim 1 wherein the anthropometric measurement includes a value of the user's height and/or waist measurement derived from an image of the user.

3. A method according to claim 1 wherein the value of the bio-impedance measurement includes a value obtained from a bio-impedance sensor.

4. A method according to claim 3 wherein the BMR estimation for the user is derived from the bio-impedance measurement.

5. A method according to claim 2 wherein deriving the value of the height and/or waist measurement from sensed image information image for the user comprises:
obtaining an image of the user's body; and
processing image information for the image to derive the value of the height and/or waist measurement.

6. A method according to claim 1 wherein the energy requirement estimation is obtained as one of:
the basal metabolic rate estimation;
a resting metabolic rate (RMR) estimation; or
a total energy expenditure (TEE) estimation derived from the basal metabolic rate estimation.

7. A method according to claim 1 further including:
assigning a body shape classification attribute to the user based on one or more of the values of biological characteristics of the set;
retrieving a template including plural fields for caloric parameters and/or macronutrient, parameters and/or expressions, said retrieved template depending on the body shape classification attribute;
wherein determining the personalised caloric and/or macronutrient intake profile for the user based on at least the energy requirement estimation comprises populating the retrieved template with caloric and/or macronutrient values, or ranges of such values, depending on the estimated energy requirement.

8. A method according to claim 7 wherein determining an energy requirement estimation further comprises adjusting the energy requirement estimation determined based on at least the basal metabolic rate (BMR) estimation depending on one of plural selectable activity level classifications classifying the user's activity level.

9. A method according to claim 8 wherein determining an energy requirement estimation further comprises adjusting the energy requirement estimation determined based on a selected one of plural goals or objectives for the user, each said goal or objective being a goal or objective associated with implementing diet management.

10. A method according to claim 9 wherein each selectable template has a predefined relationship with an associated particular body shape classification attribute, activity level classification and/or goal or objective.

11. A method for providing a personalised caloric and/or macronutrient intake profile for a user, comprising:
obtaining a set of values of biological characteristics for the user, wherein the set includes at least one value derived from sensed information, wherein the at least one value includes a value of an anthropometric measurement and/or a value of a bio-impedance measurement;
assigning a body shape classification attribute to the user based on one or more of the values of biological characteristics of the set;
retrieving a template including plural fields for caloric parameters and/or macronutrient parameters and/or expressions, said retrieved template depending on the body shape classification attribute;
determining a basal metabolic rate (BMR) estimation for the user based on the set of values of biological characteristics;
determining an energy requirement estimation based on at least the basal metabolic rate (BMR) estimation; and
determining the personalised caloric and/or macronutrient intake profile for the user based on at least the energy requirement estimation, wherein determining the personalised caloric and/or macronutrient intake profile for the user based on at least the energy requirement estimation comprises populating the retrieved template with caloric and/or macronutrient values, or ranges of such values, depending on the estimated energy requirement.

12. A system for providing a personalised caloric and/or macronutrient intake profile for a user, comprising:
input means for obtaining a set of values of biological characteristics for the user, wherein the set includes at least one value derived from sensed information, wherein the at least one value includes a value of an anthropometric measurement and/or a value of a bio-impedance measurement;
processing means for:
determining a basal metabolic rate (BMR) estimation for the user based on the set of values of biological characteristics;
processing means for determining an energy requirement estimation based on at least the basal metabolic rate (BMR) estimation; and
processing the estimated energy requirement to select the personalised caloric and/or macronutrient intake profile for the user.

13. A system for providing a personalised caloric and/or macronutrient intake profile for a user, comprising:
input means for obtaining a set of values of biological characteristics for the user, wherein the set includes at least one value derived from sensed information, wherein the at least one value includes a value of an anthropometric measurement and/or a value of a bio-impedance measurement;
one or more processing means for:
assigning a body shape classification attribute to the user based on one or more of the values of biological characteristics of the set;
retrieving a template including plural fields for caloric parameters and/or macronutrient parameters and/or expressions, said retrieved template depending on the body shape classification attribute;
determining a basal metabolic rate (BMR) estimation for the user based on the set of values of biological characteristics;
determining an energy requirement estimation based on at least the basal metabolic rate (BMR) estimation; and
determining the personalised caloric and/or macronutrient intake profile for the user based on at least the energy requirement estimation,
wherein determining the personalised caloric and/or macronutrient intake profile for the user based on at least the energy requirement estimation comprises populating the retrieved template with caloric and/or macronutrient values, or ranges of such values, depending on the estimated energy requirement.

14. A system for generating a personalised caloric and/or macronutrient intake profile for a user, comprising:
a user device for receiving input information including:
information from which the user's body shape, height, weight, age, and gender attributes may be obtained or derived; and
one or more activity attributes classifying a dominant physical activity level and/or activity type for the user;
a host server in data communication with the user device to:
receive the input information;
process the input information to:
assign a body shape classification attribute to the user;
retrieve a template including plural fields for caloric parameters and/or macronutrient parameters, said retrieved template depending on the body shape classification attribute and the one or more activity attributes;
determine a base basal metabolic rate (BMR) estimation for the user based on the derived or obtained height, weight, age, and gender attributes; determine an energy requirement estimation based on at least the determined base basal metabolic rate (BMR) and the one or more activity attributes; and
generate the personalised caloric and/or macronutrient intake profile for the user based on at least the energy requirement estimation, wherein generating the personalised caloric and/or macronutrient intake profile for the user based on at least the energy requirement estimation comprises populating the retrieved template with caloric and/or macronutrient values, or ranges of such values, depending on the estimated energy requirement; and
communicate the determined personalised caloric and/or macronutrient intake profile to the user device.

15. A system according to claim 14 wherein at least one of the body shape, height, weight, age, and gender attributes are derived from image information obtained from an image capturing device, said image information comprising one or more images of the user.
